(19) United States
(12) Patent Application Publication
(10) Pub. No.: US 2026/0041912 A1
(43) Pub. Date: Feb. 12, 2026

(54) Diagnostic Methods and Therapeutic Exercises and Equipment

(71) Applicant: Terry William Burton Moore, Guelph (CA)

(72) Inventor: Terry William Burton Moore, Guelph (CA)

(21) Appl. No.: 19/066,408

(22) Filed: Feb. 28, 2025

Related U.S. Application Data
(63) Continuation-in-part of application No. 18/801,589, filed on Aug. 12, 2024, now abandoned.
(60) Provisional application No. 63/765,025, filed on Feb. 28, 2025.

(51) Int. Cl. A61N 1/36; A61N 1/04; G16H 10/60; G16H 20/30; G16H 50/20

(52) U.S. Cl. CPC A61N 1/36034; A61N 1/0452; A61N 1/36025; G16H 10/60; G16H 20/30; G16H 50/20

(57) ABSTRACT

Methods and apparatus of diagnosing and treating a patient with a physical disorder caused by an impingement condition of either a nerve or a blood vessel. A cluster of symptoms is identified based on patient medical information and an examination of the patient. The cluster of symptoms is recognized to be related to the impingement condition. A synthetic stimulatory neural signal sequence in conjunction with one or more therapeutic motions are prescribed to eliminate the impingement condition and heal the patient.

### CROSS REFERENCE TO RELATED APPLICATIONS

This application claims priority from and incorporates by reference in its entirety U.S. patent application Ser. No. 18/801,589 filed Aug. 12, 2024, and incorporates by reference in its entirety U.S. provisional patent application Ser. No. 63/765,025 filed Feb. 28, 2025.

### BACKGROUND

### Field of the Invention

The present invention relates to health care methods and apparatus. More particularly, the

present invention relates to methods and apparatus for diagnosing and treating physical disorders due to impingement of nerves or blood vessels.

### Description of Prior Art

Various ailments and injuries lead to muscle tension or even involuntary muscle contractions. Muscle tension and involuntary muscle contraction can be overcome in some instances through massage therapy or with pharmaceutical muscle relaxants. Other ailments and injuries can lead to muscle paralysis or loss of control. Atrophy due to paralysis or loss of control can, to some extent, be treated by working the muscles, that is, by causing the muscles to repeatedly contract and relax. However, in some situations muscles cannot be worked due either to involuntary muscle contractions e.g., muscle spasms—or due to loss of control or paralysis in one or more muscles—e.g., muscle paralysis. In such instances electrical stimulation signals to work the muscles or to treat the underlying condition.

Electrical stimulation signals can be applied to selected muscles at a predefined frequency and amplitude to contract and the work the muscles. A conventional system for stimulating muscles with electrical signals is described in U.S. Pat. No. 4,838,272 to Lieber. The Lieber document describes an apparatus for applying electrical stimulation signals to selected muscles at a predefined frequency and amplitude to contract and the work the muscles to overcome muscle atrophy due to long term paralysis or inactivity. U.S. Pat. No. 6,865,423 to Oldham describes an apparatus with two electrodes that creates an electrical field within a to contract the muscle to produce induced contraction of the muscle.

### SUMMARY OF THE INVENTION

The present inventor recognized a need for methods and apparatus to diagnose and treat impingement related physical disorders. The inventor's diagnostic and therapeutic approach involves a rehabilitation model for the treatment of various musculoskeletal, myofascial and neurological conditions. It is a therapeutic model that depicts the common causality or presence of various symptoms being attributed to the paraspinal musculature. The therapeutic model involves the application of the MyoWorx® TM20 biphasic pulse generation device on the paraspinal muscles, combined with a specific approach of stretching and strengthening these and other muscles responsible for spinal posture and core stability.

### DESCRIPTION OF THE DRAWINGS

The accompanying drawings, which are incorporated in and constitute part of the specification, illustrates various embodiments of the invention. Together with the general description, the drawings serve to explain the principles of the invention. In the drawings:

FIG. 1A depicts a biphasic pulse generation device, according to various embodiments.

FIG. 1B depicts the user interface section of the biphasic pulse generation device, according to various embodiments.

FIG. 1C depicts the instrument display section of the biphasic pulse generation device, according to various embodiments.

FIG. 1D depicts a block diagram of the biphasic pulse generation device, according to various embodiments.

FIG. 2 depicts a flowchart of a method of diagnosing and treating physical disorders due to impingement of nerves or blood vessels, according to various embodiments.

FIG. 3 depicts the knee to chest exercise and stretch for treating physical disorders, according to various embodiments.

FIG. 4 depicts the seated twist exercise and stretch for treating physical disorders, according to various embodiments.

FIG. 5 depicts the seated toe touch without ankle grab exercise and stretch for treating physical disorders, according to various embodiments.

FIGS. 6A-B depict the seated toe touch with ankle grab exercise and stretch for treating physical disorders, according to various embodiments.

FIGS. 7A-B depict the seated pelvic tilt exercise and stretch for treating physical disorders, according to various embodiments.

FIGS. 8A-B depict the standing toe touch exercise and stretch for treating physical disorders, according to various embodiments.

FIG. 9 depicts the standing toe touch with leg grab exercise and stretch for treating physical disorders, according to various embodiments.

FIGS. 10A-F depict the sciatic stretch and exercise for treating physical disorders, according to various embodiments.

FIGS. 11A-B depict the lying pelvic tilt exercise and stretch for treating physical disorders, according to various embodiments.

FIGS. 12A-E depict the lower abdominal crunch exercise and stretch for treating physical disorders, according to various embodiments.

FIGS. 13A-D depict the forward rolls exercise and stretch for treating physical disorders, according to various embodiments.

FIGS. 14A-K depict the trap resistance exercise and stretch for treating physical disorders, according to various embodiments.

FIGS. 15A-B depict the side bend with head in front of shoulder exercise and stretch for treating physical disorders, according to various embodiments.

FIGS. 16A-C depict the cross arm myofascial tension (MFT) exercise and stretch for treating physical disorders, according to various embodiments.

FIGS. 17A-C depict the cross arm upper fiber trap (UFT) exercise and stretch for treating physical disorders, according to various embodiments.

FIGS. 18A-B depict the unilateral upper fiber trap (UFT) exercise and stretch for treating physical disorders, according to various embodiments.

FIGS. 19A-B depict the unilateral myofascial tension (MFT) exercise and stretch for treating physical disorders, according to various embodiments.

FIGS. 20A-B depict the lateral hip flexor tension (LFT) exercise and stretch for treating physical disorders, according to various embodiments.

FIGS. 21A-D depict the elbow circles up to forehead exercise and stretch for treating physical disorders, according to various embodiments.

FIGS. 22A-D depict the neck side bend exercise and stretch for treating physical disorders, according to various embodiments.

FIGS. 23A-G depict the sternocleidomastoid (SCM) muscle exercise and stretch for treating physical disorders, according to various embodiments.

FIGS. 24A-D depict the chin retractions exercise and stretch for treating physical disorders, according to various embodiments.

FIGS. 25A-E depict the splenius capitis exercise and stretch for treating physical disorders, according to various embodiments.

FIGS. 26A-F depict the 20-40-60 exercise and stretch for treating physical disorders, according to various embodiments.

FIGS. 27A-E depict the chin retraction and rotation exercise and stretch for treating physical disorders, according to various embodiments.

FIGS. 28A-H depict the neck isometrics and isokinetics exercise and stretch for treating physical disorders, according to various embodiments.

FIGS. 29A-D depict the Y and T shoulder exercise and stretch for treating physical disorders, according to various embodiments.

FIGS. 30A-C depict the pull down exercise and stretch for treating physical disorders, according to various embodiments.

FIGS. 31A-D depict the shoulder shrug exercise and stretch for treating physical disorders, according to various embodiments.

FIGS. 32A-M depict the shoulder shrug exercise and stretch for treating physical disorders, according to various embodiments.

### DETAILED DESCRIPTION

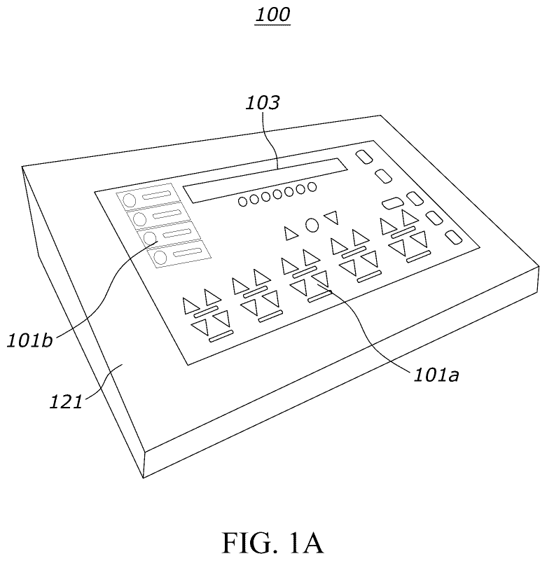

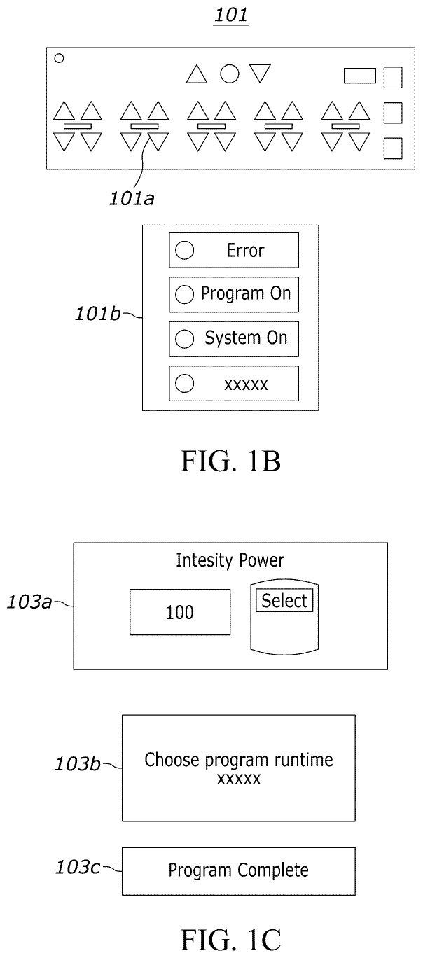

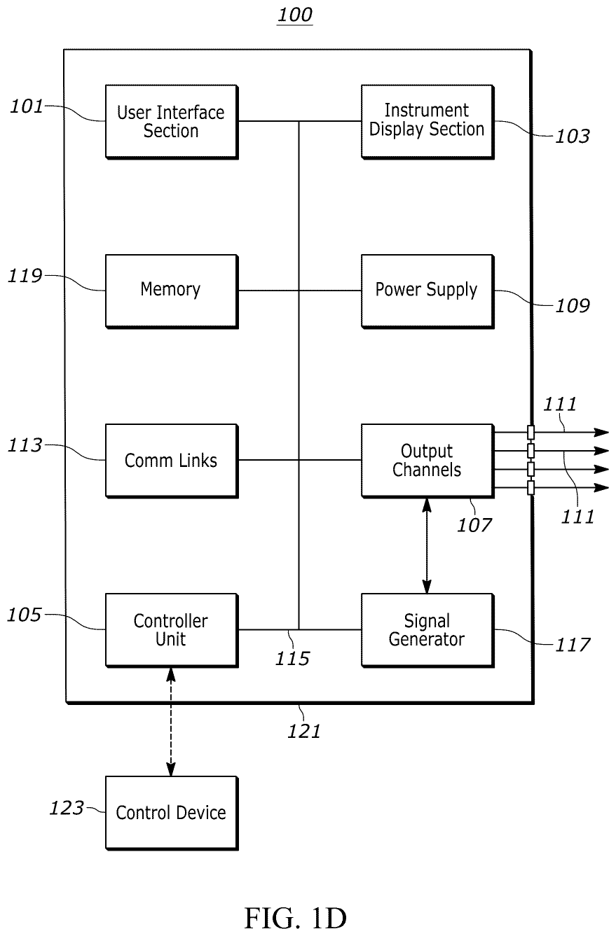

FIG. 1A depicts a biphasic pulse generation device 100 for use as therapeutic equipment, according to various embodiments. The TM20 biphasic pulse generation device 100 was developed by the present inventor. The biphasic pulse generation device 100 is used to induce muscle relaxation by applying pulses of low-level current to targeted distressed muscles or muscles otherwise causing impingement issues in the patient. By delivering a specified sequence of timed and abrupt low current level electrical pulses of various frequencies, the biphasic pulse generation device 100 is used to decrease the tension of the muscle fibers. This, in turn, leads to greater blood flow. Application of the TM20 biphasic pulse generation device 100 provides an electrotherapeutic process delivering a specific pattern of electrical stimulation that produces essential physiological responses of the nerves, muscle and the vasculature, at the cellular level. Applying TM20 biphasic pulse generation device 100 to the paraspinal muscles revealed a model for rehabilitation that has proven to be an essential diagnostic and treatment strategy for numerous musculoskeletal, myofascial, neurological and systemic pain syndromes and conditions. The specified sequence of timed low current level electrical pulses is implemented in periods of several various frequencies of between 10 hertz to 5 kHz. For example, in some embodiments the TM20 biphasic pulse generation device 100 may be programmed to provide a treatment time of 30 minutes which consists of 80 +/−20 hertz for 15 minutes, 4 +/−2 hertz for 4 minutes, 30 +/−10 hertz for 5 minutes, 50 +/−15 hertz for 2 minutes, 80 +/−20 hertz for 2 minutes and 120 +/−30 hertz for 2 minutes. In other embodiments, the frequencies and the sequence thereof for a total treatment time of 30 minutes for each circuit is 80 +/−20 hertz for 5 minutes, 120 +/−30 hertz for 10 minutes, 4 +/−2 hertz for 4 minutes, 30 +/−10 hertz for 5 minutes, 50 +/−15 hertz for 2 minutes, 80 +/−20 hertz for 2 minutes and 120 +/−30 hertz for 2 minutes. The times are preferred times, but other times are suitable as well.

The present inventor noted that fatigue or injury experienced in the paraspinal muscles is typically the causative or sustaining mechanism of various pain and symptoms of musculoskeletal, neurological and myofascial conditions. Treating fatigued or injured paraspinal muscles using the embodiments of the MyoWorx® approach positively affects various musculoskeletal, myofascial and neurological pain symptoms and conditions—particularly those that are of a chronic or recurring nature. This approach includes the use of the TM20 biphasic pulse generation device followed by a combination of muscle relaxation/release techniques and specific exercises. This approach has a substantial impact on the symptoms of some Neurological conditions that are thought to be only centrally mediated and influenced. Some of the effects are blood flow (cardiovascular) changes such as the impacts on brain fog, exhaustion and poor memory. Blood flow changes also affect hormone regulation. The low level current produced by the TM20 biphasic pulse generation device 100 can be adjusted in intensity to current levels of from 100 microamps to levels of up to 50 milliamps.

FIG. 1B depicts an embodiment of the user interface section 101 of the biphasic pulse generation device 100. In various embodiments the user interface section 101 received control inputs from a human user to adjust and operate the TM20 biphasic pulse generation device 100. In the implementation of FIG. 1B the user interface section 101 includes keypad overlays 101a-b. The keypad overlays 101a-b are typically implemented as one or more flexible membranes that cover buttons for controlling and operating the biphasic pulse generation device 100. Some of the keypad overlays 101a-b of FIGS. 1A-B have indicator lights (e.g., LEDs) next to them to indicate the state of the control switch. The keypad overlays 101a-b may provide graphic indicators—e.g., arrows—as well as labels to aid and inform the user about the operation of each button or switch. The user may operate the biphasic pulse generation device 100 by manipulating the controls shown on the keypad overlays 101a-b. The keypad overlays 101a-b allow the user to set the controls to adjust the various parameters and settings of the device, including for example, adjusting the treatment length, the intensity of each stimulation output, the maximum power of stimulation, as well as manually starting, pausing and stopping the treatment. For example, each channel has a pair of arrows—an up arrow and a down arrow—to increase or decrease the intensity of the signal on that channel. In other implementations the user interface section 101 may include one or more—or a combination of—buttons, toggles, switches, sliders, rotary dials, or other like types of user controls known to those of ordinary skill in the art.

FIG. 1C depicts the instrument display section 103 of the biphasic pulse generation device 100, according to various embodiments. The values being set, pending stage of the treatment, and/or instrument readings may be displayed to the user by means of one or more display components of the instrument display section 103. In the implementation depicted in FIG. IC the instrument display section 103 includes a numeric LED display 103a and LCD displays 103b-c. The numeric LED display 103a provides an indication the intensity of a given channel. The control for scrolling through the channels is next to the numeric LED display 103a. Instrument displays 103b-c are embodied as LCD displays that show instructions, status, error messages and other information for setting up and operating the biphasic pulse generation device 100. The instrument display section 103 may be implemented using various components and/or devices, as are known to those of ordinary skill in the art. The instrument displays 103a-c may take the form of one or more of an LCD display, alpha numeric displays, LEDs, indicator lights, or other like types of instrument screens or display components.

FIG. 1A depicts an implementation of the biphasic pulse generation device 100 that uses keypad overlays 101a-b, allowing a user to control and adjust the device. The instrument displays 103a-c show the various settings, readings and other information about the device. In this implementation the user controls and displays are integrated into the device itself. However, in some embodiments the biphasic pulse generation device 100 may be controlled by a user using a control device such as a smartphone, a laptop, a tablet, or a dedicated control device with a microprocessor or specialized control circuitry. Typically, the control device (e.g., smartphone, laptop, tablet, etc.) rests on the surface of the enclosure and connects to the biphasic pulse generation device 100 by means of a USB cable, Bluetooth or other electronic or wireless connection. However, the control device may be located remotely and communicate via wired and/or wireless communication. The control capability is typically implemented in hardware, but may in some embodiments be implemented in software or dedicated firmware. The control device may also be used to display and record settings, readings or other information about the biphasic pulse generation device 100.

FIG. 1D depicts a block diagram of the biphasic pulse generation device 100, according to various embodiments. The biphasic pulse generation device 100 typically includes a user interface section 101, a user display section 103, a controller unit 105, one or more output channels 107, a power supply 109, stimulation cables 111, comm links 113 internal connections 115, signal generator(s) 117 and an enclosure cabinet 121. As discussed above, the user interface section 101 may be implemented as keypad overlays 101a-b or other types of switches or input components. The user display section 103 may be implemented as a numeric LED display 103a, LCD displays 103b-c and indicator lights, or other types of display components.

In some embodiments the controller unit 105 is implemented internally, inside the enclosure cabinet 121. The controller unit 105 may include one or more microprocessors, dedicated control circuitry, or other electronic logic hardware, software or firmware either in combination or by themselves, as is known to those of ordinary skill in the art. The electronic logic hardware of controller unit 105 controls the functions of the biphasic pulse generation device 100 in response to inputs received from the user. In some embodiments the controller unit 105 may be in communication with, or embodied as, an external control device 123 such as a smartphone, a laptop, a tablet, or a dedicated control device with a microprocessor, specialized control circuitry or other type of logic components. In some embodiments an external control device 121 may serve as the controller unit 105 to operate the biphasic pulse generation device 100.

The enclosure cabinet 121 may be embodied as a custom bent metal enclosure. Alternatively, the enclosure cabinet 121 may be made from plastic or other synthetic material with suitable strength and weight characteristics, as known by those of ordinary skill in the art. In some embodiments the enclosure cabinet 121 may be waterproof to prevent moisture on the inside of the cabinet, while in other embodiments the enclosure cabinet 121 may be vented to provide air flow within the cabinet to keep the power supply and electronic components cool. The power supply 109 is typically an AC power supply positioned within the enclosure cabinet 121 and provided with a corded plug compatible with a standard wall outlet, e.g., 120 volts in the U.S. and Canada. In some embodiments the power supply 109 may be located outside the enclosure cabinet 121 and connected to the internal electronic components via wired connections. In some embodiments the power supply 109 may be a DC power supply (e.g., a battery) to enable mobile operation of the biphasic pulse generation device 100.

Stimulation cables 111 include the cables that connect the patient to the biphasic pulse generation device 100. Typically, the stimulation cables 111 are flexible insulated electrical cables between two and fifteen feet long with a connector at one end and a patch electrode at the other end. The connector is attached to the biphasic pulse generation device 100, and the patch is typically placed on the patient's skin with an adhesive. The stimulation cables 111 are provided with one or more electrodes at one end which each fasten to the patient's skin near a distressed muscle, and a plug at the other end. Typically, each stimulation cable 111 has two electrodes—but in some implementations may have multiple electrodes. The end of each stimulation cable 111 opposite the electrode(s) is configured with a plug that is compatible with sockets on the TM20 biphasic pulse generation device 100. The electrodes are temporarily secured to the patient using straps, adhesive surfaces, suction cups or other like types of securing means. The electrodes are “temporarily” connected inasmuch as they are configured to be connected during the treatment and then disconnect without harming the patient immediately following the procedure.

Comm links 113 include the communication links that run between the biphasic pulse generation device 100 and a control device 199 or other devices. The comm links 113 may be either wired links—i.e., cables or wiring—or wireless links such as Bluetooth, cellular telephony signals or other radio waves. In some embodiments comm links 113 an Ethernet interface or other communication electronics for communicating via the Internet or a private network.

The TM20 biphasic pulse generation device 100 is typically designed, assembled, and tested to the medical device standards and all safety and regulatory requirements of the various countries in which it is intended to be used. For example, all TM20 biphasic pulse generation devices 100 sold and used in Canada conform to the standards of a Class 2 device as required for Health Canada and the FDA. The device is designed to operate in the temperature range of from 10-35 degrees Celsius.

In various embodiments, the device is programmed to apply the pulses of low level current at various frequencies in 20 second bursts, with 0.5 seconds between bursts. In some implementations the bursts may be as short as 3 seconds to as long as two minutes, and the rest period between bursts may be as short as 0.1 second to as long as 10 seconds. In one embodiment there are a series of 20 second trains of electrical impulses at a particular frequency with a 0.5 second gap between each train. The pulse width of the low level current pulses may be varied to values of from 1 microsecond to 1 second. In one embodiment the pulse width is of 200 +/175 microseconds and the wave form is approximately rectangular in nature. In various embodiments each successive pulse is approximately the same amplitude of the previous pulse, but inverted. For example, the pulse voltage in a pulse train may be: +25V, −25V, +25V, −25V, etc. The maximum voltage is 70 volts and the minimum voltage is −70 volts with a peak at a resistance of 1 K ohm. In some implementations the voltages may be set to no greater than 20 volts or less than −20 volts. Each train represents one frequency and trains are successive.

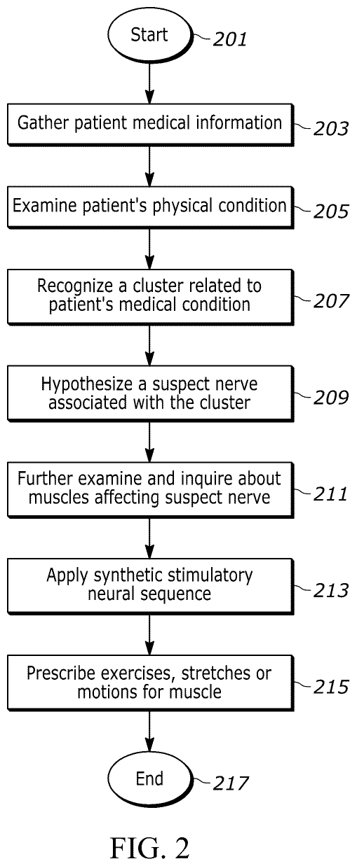

FIG. 2 depicts a flowchart of a method of diagnosing and treating physical disorders due to impingement of nerves or blood vessels, according to various embodiments. The method begins at 201 and proceeds to block 203 where patient medical information is gathered. Gathering patient medical information includes gathering information about any illness, injury or other form of bodily degradation that the patient has experienced. The process of gathering the patient's medical information is ongoing, and may coincide with other steps in the diagnosis or treatment. For example, the gathering of patient medical information also occurs during the physical examination of the patient in block 205. Thus, blocks 203-205 can be thought of as being performed simultaneously.

Typically, the process of gathering patient information in block 203 and examining the patient's physical disorder in block 205 begins by having the patient fill out a medical questionnaire as part of the process of checking in. The questions include queries about the pain or other medical symptoms the patient is experiencing. The patient may also be weighed and measured at this time. The gathering of medical information may also involve a brief interview or discussion with the patient to get a more comprehensive explanation of the physical disorder for which treatment is being sought. For example, the patient would be asked whether they've had any fractures, surgeries, illnesses, falls, motor vehicle accidents (MVAs), and whether or not the patient experienced a known issue from any of these conditions or any previous injuries that stand out in the patient's mind. In addition, the patient's medical records may be obtained to determine the patient's medical history and ascertain whether there are other related factors or causes that may contribute to the physical disorder.

The patient may be asked the following questions or similar inquiries as part of gathering the patient's medical information: What is the main problem or physical disorder that they are here for? When did the physical disorder start? What part of the body did it seem to start in? Is this the first time the patient has had this problem? If no, how many times has it occurred and when was the first time? What was the patient doing at the time the physical disorder first presented? Does the patient have life stressors either now or in the past? What are the specific symptoms of the physical disorder? The patient may be asked to list specific detailed descriptions of symptomology. Is the pain, if any, sharp, dull, burning, aching? Are there sensations of fatigue—that is, local muscle tiredness/lack of endurance—versus a feeling of heaviness—that is, motor nerve root compression? Since fatigue tends to clear up faster than heaviness, has this happened in the past and gone away? Does the patient have any history of para-spinal issues within and around innervation segment levels related to the symptoms? Does the patient have symptoms to be included in symptom clusters?

A given physical disorder caused by muscle impingement on one or more nerves or vessels tends to cause a clusters of symptoms which may be called a symptom cluster. Therefore, knowledge of various symptom clusters is useful in diagnosing an impingement related physical disorder. Gathering the patient's medical information includes specific inquiries as to whether the patient has experienced any of the following symptoms which are commonly found in symptom clusters: dizziness, blurred vision, nausea, bowel/bladder issues, tinnitus, VA, ISA, IVA, problems swallowing, heartburn, loss of consciousness, plugged cars, TMJ dysfunction and whether the patient wears any orthotics.

The gathering of patient medical information may include the following specific inquiries about each of the symptoms often present in symptom clusters. If dizziness presents, the patient should be asked whether it's dizziness (i.e., feels like they are moving) or vertigo (i.e., feels like the room is moving) or imbalance (i.e., feels off-balance as if they could fall to one side or other). If the patient has blurred vision, inquiries should be made to determine whether vision is blurred due to astigmatism or not. Determine whether there are any visual disturbances such as aura, double vision, floaters or spots in their field of vision. Determine the frequency of nausea and whether anything precedes or prompts the condition—e.g., eating or smelling something, heights or motion. Determine whether bowel or bladder irregularities are related to frequency, urgency, leaking, constipation or typical stool type. For tinnitus note whether it is a high or low pitch tone, humming, ringing or whistling, the common variations of tinnitus. Muffled hearing or water in the cars or plugged cars are related to blockage or damage of the eustachian tube and are not tinnitus. Determine whether there is pain with coughing/sneezing or bowel movements/urination. Note the location of any pain. For problems swallowing, find out if there is pressure in the patient's throat or the feeling of something getting stuck partway down. Note what level the feeling of stuck is and if particular foods or liquids make it more problematic to swallow. If the patient has heartburn, determine whether there is a persisting mild dry cough or the typical burning pain of heartburn. Confirm any loss of consciousness by a loss of recollection of a period of time. If the patient has plugged cars, confirm that it is Eustachian tube related, and not a type of tinnitus. If there is Temporomandibular joint dysfunction (TMJ dysfunction), inquire about pain (with or without palpation), slide/shift, decreased mouth opening, locking (open or closed), clicking or popping. Inquire as to whether the patient wears any orthotics?

The patient should be asked about any other symptoms in a suspected symptom cluster at this time, such as tenderness or tension in the wrist extensors, wrist flexors, thenars, anterior deltoids, vastus medialis oblique muscle (VMO), lateral head of gastrocnemius, medial head of gastrocnemius muscle, tibialis posterior and soleus muscles, tibialis anterior, peroneal nerves and plantar fascia. Another aspect of gather medical information involves making postural observations from the front, side and back as well as observations of the positions/angles of the head, shoulders, spine, feet, hands and eyelids. In addition, the facial muscles should be checked for tone tenderness and tension. Various hand grip tests can be performed to gather further patient medical information. For example, the patient's hand grip strength should be tested while patient perform is sitting, with patient's elbow flexed to 90 degrees, with the forearm parallel to floor (and not resting on armrest). Hand grip force can be measured with a dynamometer with the patient in various postures and positions. Typically, hand grip is tested by having the patient squeeze as hard as they can a number of times—e.g., three times for each hand, alternating hands between tests.

Typically, a number of tests and exercises are performed to test core strength. The quarter-crunch test involves crossing the patient's arms over their chest with hands on shoulders while lying on the examination table. The patient then rolls their head and shoulders up so the shoulder blades come off table, holds the position, continuing with regular breathing. If the patient reports pain, discomfort or symptoms, they are to stop as needed. After performing the quarter-crunch test for approximately 45 seconds note any shakiness, pain, fatigue or other complaints. The supine bilateral leg raise is performed by having the patient cross their arms over chest with hands on shoulders then, keep their knees straight, the patient lifts both legs off table approximately six inches while breathing regularly. If the patient reports pain, discomfort or other symptoms, they are to stop as needed. After performing the supine bilateral leg raise approximately 45 seconds note any shakiness, pain, fatigue or other complaints. The prone torso lift test is performed by having the patient place a pillow under their hips while on the examination table, with their hands resting on small of their back and palms facing upward. With legs relaxed, the patient is to lift their head and chest off the table and hold, while breathing regularly. Care should be taken to avoid neck extension. If the patient reports pain, discomfort or other symptoms, they are to stop as needed. After performing the prone torso lift approximately 45 seconds note any shakiness, pain, fatigue or other complaints. The prone leg lift test involves the same position with a pillow as with the prone torso lift. Keeping their head down and knees straight, the patient is to lift both legs in their entirety off table. Check to ensure the patient's knees and lower thighs have cleared table. If the patient reports pain, discomfort or other symptoms, they are to stop as needed. After performing the prone leg lift approximately 45 seconds note any shakiness, pain, fatigue or other complaints.

The glut activation test (sometimes called single straight-leg bridge) is performed by holding a bridge and then immediately extend one leg straight maintaining alignment with other thigh while continuing to breath regularly. This should be done for 10-15 seconds, and then repeated with the other leg. During this exercise check which muscles are working (e.g., gluts/hams) and verify stability by applying slight downward pressure on the opposite hip. Observe and note any wobbling, shaking, or complaints of pain from the patient. One of the manual muscle tests to be performed is the isometric neck strength test. An instrument such as an echo Commander™ Manual Muscle Tester (MMT) can be used to take measurements while exercising the neck to exert force against a fixed resistance such as a wall. Neck force should be applied in four directions with measurements being made in each direction: flexion, extension, right and left lateral flexion. The Balance Error Scoring System (BESS) balance test and the treadmill stress test are two other tests that may be performed to gather further patient medical information. The Snellen Eye Chart test or other similar eye test may be used to test the patient's eyes. For the Snellen Eye Chart test the patient stands with toes at a line marked on floor, covering one eye (don't close eye), and reads the smallest line that can easily seen. If one or more errors occur in a line, the previous line is recorded as being read without errors. The patent then repeats this process with other eye. The test is performed both with glasses on and glasses off.

Upon completing the gathering patient information in block 203 and examining the patient's physical disorder in block 205, the method proceeds to block 207. In block 207 the practitioner evaluates the symptoms of the patient to determine whether the patient has any symptom clusters. There are two classifications of symptoms in symptom clusters—core symptoms (CS) and condition indicator symptoms—which may be called condition indicators (CI). Core symptoms are stronger indicators of a physical disorder than are condition indicator symptoms. Each physical disorder generally has one or more core symptoms associated with it. At least one of the associated core symptoms will always be present for a given physical disorder. Condition indicator symptoms, on the other hand, may also be present for a given physical disorder. Sometimes, however, a patient may have a physical disorder even though the patient does not exhibit all the associated condition indicator symptoms. Hence, the condition indicator symptoms are an indicator of the physical disorder, but not as strong of an indicator as the core symptoms associated with the physical disorder. Table I, below, lists the core symptoms and condition indicator symptoms for post concussion syndrome (PCS), temporal mandible disorders (TMD), migraines/headaches, Parkinson's disease (PD) and glaucoma. Once any symptom clusters have been identified for the patient in block 207 the method proceeds to block 209.

In block 209 the practitioner hypothesizes a suspected impingement condition based on the identified symptom cluster, the collected medical information of the patient, and the examination of the patient. The hypothesized impingement condition may be a physical disorder such as a nerve impingent—e.g., a muscle exerting pressure on a nerve affecting to the point that it causes pain or affects the nerve function. Another type of hypothesized impingement condition may be a physical disorder such as a blood vessel impingent—e.g. a blood vessel being impinged upon by muscles, tendons or bones in a manner that restricts or otherwise affects the blood flow. Once the practitioner has hypothesized a suspected impingement condition in block 209 the method proceeds to block 211.

In block 211 the practitioner further examines the patient to verify the hypothesized impingement condition and determine whether any more symptoms exist in the patient. It sometimes happens that a patent will overlook symptoms that are not particularly troublesome, and only report those that cause considerable pain or diminished physical abilities. The further examination of the patient in block 211 may also allow the practitioner to more carefully feel the area of the hypothesized impingement, and possibly verify the impingement through feel or observation of swelling, redness, etc. Once the practitioner completes the further examination of the patient in block 211 the method proceeds to block 213.

In block 213 the practitioner applies a course of synthetic stimulatory neural sequences to the muscle causing the impingement condition. The TM20 biphasic pulse generation device 100 may be used to apply the therapeutic synthetic stimulatory neural sequences to the muscle. This acts to reduce swelling and halt muscle spasms, thus providing some relieve for the impingement condition. The course of synthetic stimulatory neural sequences may take place during several sessions over a period of days, or even months in some instances. In a method of operating the device, the power switch is turned on and the standby indicator light is illuminated. The electrodes on stimulation cables 111 are placed on the patient with one electrode being positioned on the patient's skin adjacent either end of the muscle to be treated. The user can more particularly be described as a patient. The electrodes are connected via stimulation cables 111 the TM20 biphasic pulse generation device 100. Upon removing the device from the standby mode and activating it, the electrodes in contact with the patient provide a low-level current flow of pulses through the muscle to be treated. Positioning multiple electrodes on the patient enables the simultaneous treatment of several muscles at a time. For example, when one or more muscles are being treated are on one side of the patient's body, the same muscles on the other side of the patient may also be treated. The operator of the device may set the treatment time for periods of from 5 to 120 minutes. In various embodiments the treatment time may be 30, 45, 60 or 90 minutes, as is deemed appropriate for a particular patient.

In various embodiments the device is programmed to proceed through different frequencies at their proper time allotments based upon the total program time chosen by the operator for that patient and inputted it into the device as stated above. The sequence of frequencies are predetermined and preferably pre-programmed into the device through a programmable controller. In various embodiments the frequencies and the sequence thereof for a total treatment time of 30 minutes for each circuit is 80 hertz for 15 minutes, 4 hertz for 4 minutes, 30 hertz for 5 minutes, 50 hertz for 2 minutes, 80 hertz for 2 minutes and 120 hertz for 2 minutes. In other embodiments, the frequencies and the sequence thereof for a total treatment time of 30 minutes for each circuit is 80 hertz for 5 minutes, 120 hertz for 10 minutes, 4 hertz for 4 minutes, 30 hertz for 5 minutes, 50 hertz for 2 minutes, 80 hertz for 2 minutes and 120 hertz for 2 minutes. The times are preferred times, but other times are suitable as well.

Upon performing the synthetic stimulatory neural sequences to the muscle causing the impingement condition, the method proceeds to block 215.

In block 215 the practitioner prescribes one or more stretches, exercises or other therapeutic motions for the patient to do. The stretches, exercises or other therapeutic motions are aimed at eliminating the impingement condition by stretching—or in some instances, repositioning—the offending muscle. The patient may be instructed in how to perform the therapeutic motions, and provided with a schedule for completing them. The patient may do the therapeutic motions during the same time frame—e.g., on the same days or within the same week—as the therapeutic synthetic stimulatory neural sequence sessions. That is, the two types of therapies may be done on the same days, or in some instances, on alternating days. Once block 215 is completed the method proceeds to block 217 where it ends.

The following table lists Core Symptoms (CS) and Condition Indicator Symptoms (CI) for the following five physical disorders: Post Concussion Syndrome (PCS), Temporal Mandible Disorders (TMD), Migraines/Headaches, Parkinson's Disease (PD) and Glaucoma.

TABLE I

| Symptom | PCS | TMD | Migraine | PD | Glaucoma |
|---|---|---|---|---|---|
| Acid Reflux | CS | | CI | | |
| Anxiety | CS | | CI | | |
| Balance Impairment | CS | | CI | | CI |
| Blurred Vision | CS | | CS | | CS |
| Brain Fog | CS | | CI | | CI |
| Depression | CS | | CI | | |
| Dizziness | CS | CI | CI | CS | CI |
| Double Vision | CI | CI | CI | | |
| Dry Eyes | CS | | CI | | CI |
| Eye Pain | CS | | CI | | CS |
| Face/Neck Pain | CI | CS | CI | | |
| Facial Masking | | | | CS | |
| Fatigue | CS | CI | CI | | |
| Insomnia | CS | CI | CI | CS | |
| Involuntary Movements | | | | CS | |
| Irritability | CS | CI | CI | | |
| Jaw Clicking/Popping | CI | CS | | | |
| Jaw Joint Pain | CI | CS | | | |
| Jaw Movement | CI | CS | | | |
| Impairment | | | | | |
| Light Sensitivity | CS | | CI | | CI |
| Migraines/Headaches | CS | CS | CS | | CS |
| Muscle Stiffness | | | | CS | |
| Nausea | CS | CI | CS | CS | |
| Ocular Pressure | CI | | CI | | CS |
| Problem | CS | | CI | | |
| Solving/Thinking | | | | | |
| Impairment | | | | | |
| Rainbow Halos | | | | | CS |
| Red Eyes | CI | | CI | | CS |
| Self-Control Problems | CS | CI | CI | CS | |
| Short Term Memory | CS | CI | CI | | |
| Impairment | | | | | |
| Smell Impairment | CS | CI | | CS | |
| Stooping/Hunched Over | | | | CS | |
| Swallowing Difficulty | CI | | CI | CS | |
| Taste Impairment | CS | | | CS | |
| Tinnitus | CS | CI | CI | | CI |
| Tremors | | | | CS | |
| Tunnel Vision | CI | | CI | | CS |
| Urinary/Bowel Problems | CI | CI | CI | CS | |
| Vertigo | CI | CS | CI | | |
| Vision Blind Spots | CI | | CI | | CS |
| Vision Impairment | CI | | CI | | CI |
| Voice Impairment - | | | | CS | |
| Soft/Low | | | | | |
| Walking Impairment | | | | CS | |
| Writing Impairment - | | | | CS | |
| Small | | | | | |

CI—Condition Indicator Symptoms
CS—Core Symptoms

FIG. 3 to FIG. 32M depict positions of various stretches and exercises for treating physical disorders, according to various embodiments.

### Lower Body Stretches and Exercises

FIG. 3 depicts the knee to chest stretch, according to various embodiments. The knee to chest stretch is one of the easiest lower back stretches to do and is also one of the most comfortable for people. It can be done sitting or lying. If someone has very severe pain in the lower back, you may have to do it lying flat on the bed in a supine position, but the stretch may actually have greater therapeutic value if done sitting in a chair.

To perform the stretch, the patient slouches in a chair by sliding his buttocks to the front and then leaning back. The patient's buttocks will be towards the very front of the chair. With one leg out straight, the patient puts his forearm under the other knee, grabs the wrist with his opposite hand and brings his knee up towards his forward-leaning head as if he is trying to hook his knee over top of his ear. The patient may not actually feel much of anything in his back when he does this, but should hold the stretch for three very slow deep breaths. Then the patient brings his leg down to relax, and alternates sides. Like most stretches, when the patient exhales, that's when the maximum stretch is reached. For patients that are very tight, the straight leg typically lifts off the ground as the stretch is done, but will gradually drop while holding the stretch. It is preferable for the patient to not arch his back when he brings the leg down. The doctor may instruct the patient to pull their stomach in tight as they bring the leg down, otherwise the lower back arches and can give the patient a jolt of pain. It is also important to instruct the patient to keep their stomach sucked in. Another thing to note, is that when the patient lifts up his right knee, for example, he will likely also feel the effect of the stretch in his left leg. For instance they'll often feel something in the quadriceps of the left leg, which is a good example that this is actually causing stretching on the opposite side. Once the patient sits up again, he will probably find his legs feel a lot looser. When the patient gets up and walks around, there will be most likely be reduced pain in the leg. Again, if there is pain in the left leg, the patient should bring his right knee up, not the left knee, which is what is typically taught. But the patient will actually sec better results if they bring up the opposite leg. If there is severe pain it will be much easier to have the patient do the knee to chest exercise lying on a bed or floor, in the same manner as it is done in a chair. The patient should be instructed to suck their stomach up into the rib cage because when they bring their leg down, they tend to arch the lower back and as a result get a jolt of pain. The knee to chest exercise is among the easiest exercises possible and very gentle when the doctor guides the patient to do it slowly and focus on the abdomen.

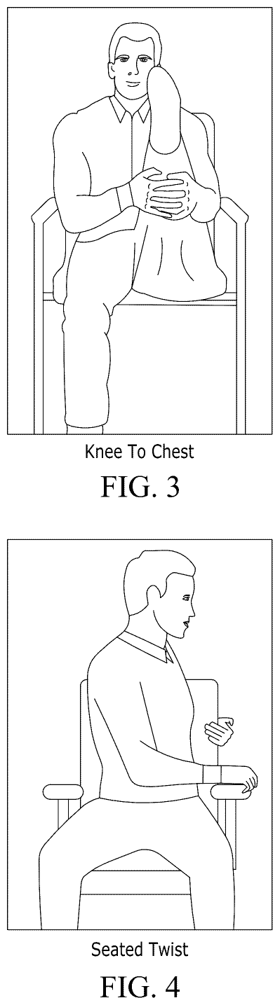

FIG. 4 depicts the seated twist stretch, according to various embodiments. Another important exercise for the lower back is the seated twist stretch exercise done in a chair. This stretch can also be done lying down, just letting the knees drop to one side. The seated twist stretch can be done anywhere, for example, in the patient's office or living room. To perform the seated twist stretch, the patient should sit up straight with his back two or three inches away from the back of the chair.

Otherwise, when the patient twists the back is going to press against the chair back and as a result, not rotate very far. The patient should sit forward in the chair with an upright posture and keep his stomach sucked in, then rotate. The patient should grab the bottom of the chair (e.g., with the right hand when rotating to the left) and the left hand would grab onto the back of the chair to twist the torso to the left as far as it can go. Again, when the patient twists the torso to the left, he pulls with the right hand (on the bottom of chair) and pushes with the left hand (on the chair back).

The patient should hold the twist for three deep breaths, then slowly rotate back. The doctor should make sure the patient is using a slow, steady pressure. If the twisting motion is done too rapidly, swinging the body around, there is a high probability of a rebound contraction in the stretched muscles and that may aggravate the injury. Do the opposite motions and grasps for the other side. Stretching away from the side (of the back or leg) that hurts tends to be the most therapeutic: “twist away from the pain.” For example, if the pain was in my left leg, the patient would preferably stretch to the right—that is, rotate to the right.

People will find quite a relief from this exercise. The doctor should take care to make sure it is done slowly and gently. The twist in the chair is quite useful for stretching the lower lumbar and upper sacrum. There aren't many other exercises that are as effective for stretching the lower lumbar and upper sacrum as the seated twist stretch, so it is very useful if there is sacral pain.

FIG. 5 depict the seated toe touch stretch (without ankle grab), according to various embodiments. The seated toe touch stretch is generally quite an easy stretch for most patients. Moreover, if the patient has a tremendous amount of pain in the back or lower back, it may be best not to attempt a standing toe touch. Instead, it is better to do a progression and same as everything else, starting with a seated toc touch stretch. The patient should suck the stomach up into the rib cage as this stretch is done, otherwise the low back arches and the patient will likely experience pain.

A lot of patients are frightened to bend forward when the back is sore. So one can start a slow progression with it by positioning the patient's hands on his thighs, having him breath in and suck the stomach up into the rib cage, and then roll down, exhaling as the patient stretches downward. The patient should slowly curl the torso down, breathing deeply in and out while bending forward and continue to support with the hands on thighs bringing the shoulders toward the knees. When the patient gets down to the bottom of the stretch, he should take one or two deep breaths. This will often enable the patient to stretch downward a little bit further.

Next the patient pulls the stomach in and uses the stomach to straighten back up. The patient should avoid using the back muscles, and instead just pull in and tighten the stomach. This will force the back to roll up one vertebrae at a time. This is the point of using the stomach to force the back to roll up one vertebrae at a time, otherwise the patient will end up with segments of the lower back that don't move. Once the patient is comfortable doing it with his hands supporting his stomach, he can progress the stretch by doing it without hand support. The patient can just let his arms hang down the front or the sides of the legs and repeat bending the torso forward as described above. The seated toc touch stretch will prepare the patient for the standing toc touch.

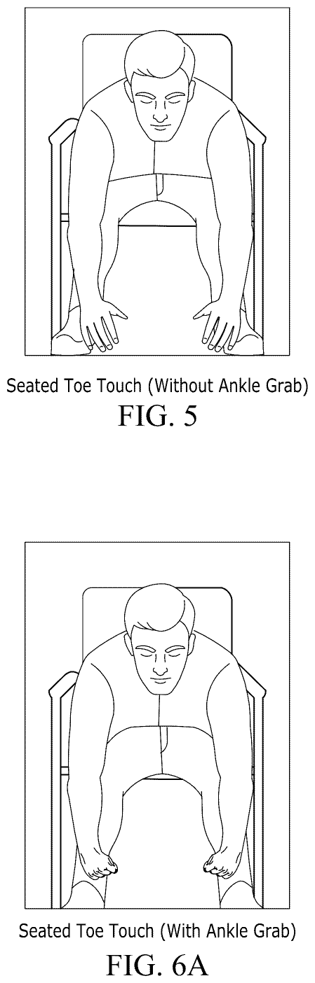

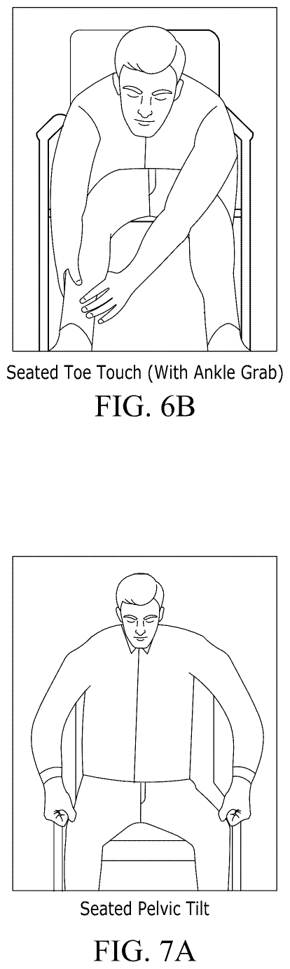

FIGS. 6A-B depict the seated toc touch stretch (with ankle grab), according to various embodiments. This exercise is a variation that can be added to the seated toe touch that leads to the standing toc touch, and can be used as a sciatic stretch for some patients who are unable to do the sciatic stretch correctly. This variation of the seated toc touch starts off the same as the seated toc touch (without ankle grab), except this time, once the patient gets down to his ankles, he grabs onto his ankles, pushing his lower back up and back towards the back of the chair. The stretch should be held for two or three deep breaths, then come up a couple of inches more, grab on the calves and do the same thing, pushing the lower back up and back towards the back of the chair, making contact higher up in the lower back. After that, come up to the knees, holding the knees and push up the back, which will be upper lumbar/lower thoracic level in this case, trying to flatten my lumbar spine against the back of the chair again holding for two to three deep breaths. This will provide tremendous flexibility and is an effective way to help patients create flexibility in a specific area of the mid-low back where they may be lacking flexibility.

The seated toe touch stretch (with ankle grab) can be varied if pain symptoms are worse on one side or the other by doing it exactly the same, but only doing it at a 45 degree angle, so that the patient has just come down to one leg (away from the side of pain). For example, grabbing onto the ankle, calf and knee on the right leg, will focus the stretch in the left side of the back. If the patient is more advanced than that and they're not frightened by the concept of doing it, a similar stretch can be done from the standing position as well.

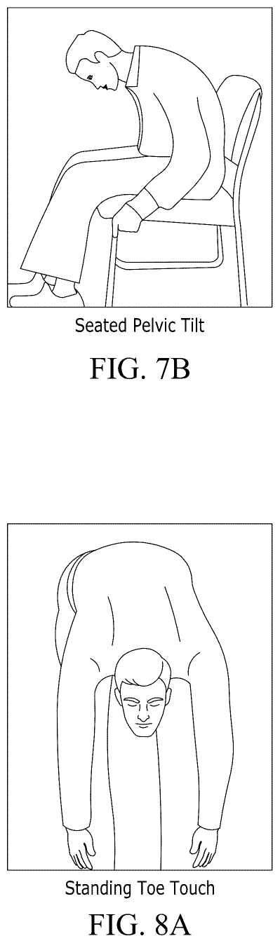

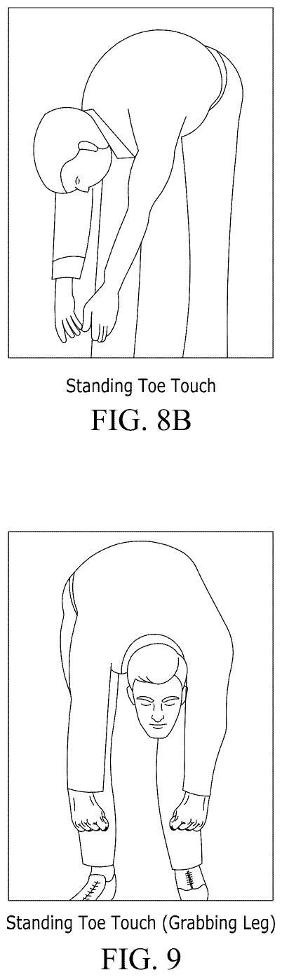

FIGS. 7A-B depict the seated pelvic tilt exercise, according to various embodiments. Patients often feel a bit tender or feel stretch discomfort after they've done several of these exercises for the lower back. The seated pelvic tilt is an effective way to help stretches the lower back with minimal stretch pain while also increasing the blood flow in the tender low back muscles. Have the patient sit back against the chair, nice and straight, and pull their stomach up into the rib cage while leaning forward—especially for men. Women can do it sitting more upright, but men generally have to be leaning forward somewhat. Women may also find it easier to do leaning forward. The patient should lean forward about 45 degrees, grab the bottom of the seat and use his arms and legs to assist.

The seated pelvic tilt exercise is not meant as a stomach strengthening exercise. It will add some strength, but not a lot. The seated pelvic tilt exercise is primarily meant for stretching to get rid of stretch discomfort in the lower back muscles. The patient should use his feet and arms to aid in pushing his back into the chair and holding it for about five to ten seconds, while breathing regularly and relaxing. As the patient presses with his feet it may feel like he is pushing his sacrum, or tailbone (coccyx), back into the chair. The patient should use his arms and feet as well as his stomach for this stretch. The patient does use his stomach, but uses other muscles as well to help in the stretch. The low back muscles generally relax, providing the patient with an almost instantaneous reduction in pain. The seated pelvic tilt exercise can also be done in a lying position, which tends to aid more in abdominal strengthening, and may not be as effective for a lower back stretch.

FIGS. 8A-B depict the standing toe touch stretch, according to various embodiments. For the standing toe touch exercise, the patient stands with his feet approximately shoulder width apart, and sucks the stomach up into the ribcage. The patient keeps the stomach sucked in during the stretch to avoid using the hips. The patient's back should slowly bend forward. The patient takes deep breaths in and out as he slowly bends forward at the waist, but not by forcing himself or letting himself flop forward. If a patient lets himself flop forward, he is at risk of pulling a back muscle.

Once the patient goes as far down as he can (hands to the floor if possible), he then pulls his stomach in and uses the stomach to raise his upper body, letting his buttocks curl in. This keeps the back rounded as the patient rolls fully upward. Otherwise, the patient will roll part way and then suddenly arch his back, and they wonder why they're sore. Typically, the patient will not realize he is using his back muscles to stand upright again. But as long as the patient concentrates on rolling upward using stomach, it forces him to roll with his stomach muscles rather than simply standing upright using his back muscles. So rolling with the stomach is actually an important part of this exercise. One final consideration is that the patient should let his knees bend slightly, rather than doing the stretch stiff legged.

One thing that can be added to this stretch is to rotate the torso while going down and back up. Doing this will provide more of a stretching benefit, and work slightly different muscles. It also increase the blood flow in the back muscles as well, so they stretch easier, while hanging down. People tend to rotate their hanging arms and forget about moving the torso, so rotating the torso ensures that the entire torso moves and stretches during the standing toe touch stretch.

Then come to the front with the torso, or if they're tighter on one side, go to a 45 degree angle with the torso, pull the stomach in, which rolls the body up again and tuck the buttocks. It's basically a continuous motion, so it's dynamic and therefore the stretch is actually far better.

FIG. 9 depicts the standing toc touch stretch (grabbing leg), according to various embodiments. As with the variations on the seated toe touch, the patient can also do the same with the standing toc touch if the person is a bit more advanced and they're not frightened of trying it. For this variation the patient sucks his stomach up to the ribcage, leans forward with arms hanging down, and positions the hands just below the knees. The patent then grabs onto his legs (below the knees) and push the lower back up.

Hold the position, the patient pushes up the lower back for about five seconds through a deep exhalation while relaxing. Then the patient drops down approximately three inches, again grabbing onto the shin and pushing up in the lower back. The doctor should advise the patient to aim for the arca that's hurting, if possible. The patient should literally be fighting the pain. The patient breathes in, exhales and pushes up the lower back to the point where it's sore. Next the patient slides his hands down, grabs his ankles, breathes in, exhales, and push up the lower back again. The patient can use the legs to help push up the lower back as well. To get back up the patient should use his stomach muscles to roll up. In doing this the patient may be able to feel each vertebrae move quite distinctly. Generally, if the patient tries to bend forward again after doing all of that, it be easy to come down again. Once the patient can stretch down near or at the floor with his hands, he can then bring in the torso rotation. This tends to loosen up the lower back muscles very quickly in most instances.

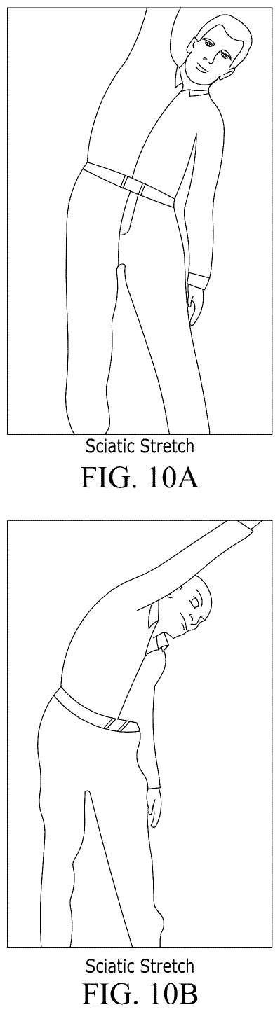

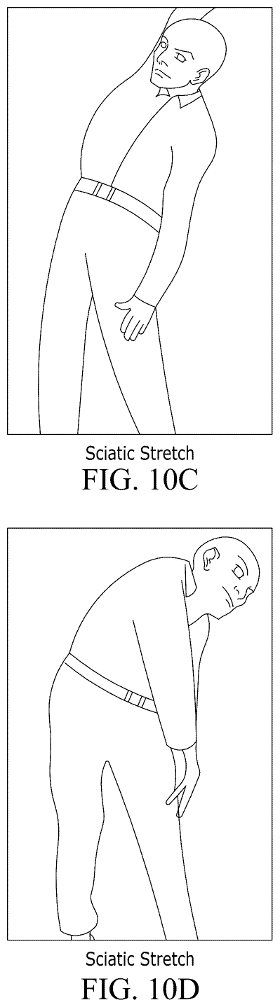

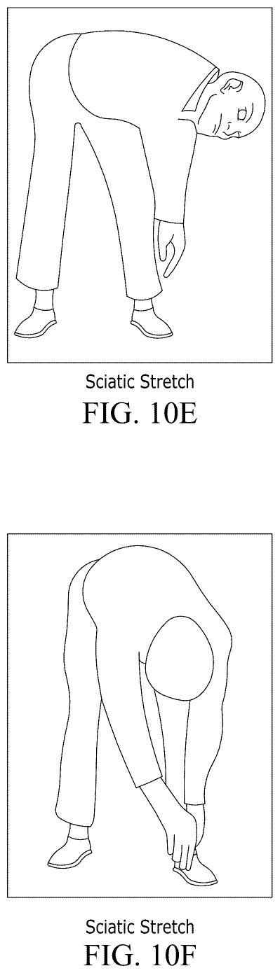

FIGS. 10A-F depicts the sciatic stretch, according to various embodiments. The sciatic stretch is perhaps the best lower back stretch, and ultimately can replace the other ones. But patients in severe pain may need to do the other lower back exercises to stretch it out so they can eventually do the sciatic stretch. To perform the sciatic stretch the patient sets up like the standing toe touch with his feet shoulder width apart, or maybe a little bit broader than shoulder width.

The patient places one foot an inch or two forward from the other. Standing up straight, the patient sucks the stomach in, and tilts the torso to the side of the foot that was placed slightly more forward, using the hand to support the torso (on the side of thigh) on the side the patient is bending towards. The patient then raises up the opposite arm over his head, so the weight of that arm causes a greater stretch in that side.

This simple exercise alone (torso side bend), that is the first part of the sciatic stretch, will get rid of IT band tightness and pain on the side you're stretching away from. Because we're targeting a broad muscle, to get all the fibers you would rotate the torso forward as if to reach forward and then backward, slowly back and forth a few times, continuing to breathe deeply. You can feel it's changing the stretch to different parts of the muscle as you rotate the torso.

After rotating the torso a few times, the patient stops at the side and brings the overhead arm around and down with the other hand supporting (on the side of the thigh) on the side the patient is bending towards. Now the patient slides both hands slowly down to the outside of the shin while taking deep breaths and keeping the stomach pulled in (otherwise it's going to be more of a hip stretch, not a lower back stretch). The patient should let the body come down to the ankle, basically trying to come down towards the little toc.

Once the patient has brought both hands down toward the outside of the ankle (on or near the floor), he continues with deep breathing and rotates his torso. The patient can imagine tracing out from the little toc around to the arch of the foot using the hands (slowly rotating back and forth around the outside to inside of the foot). This movement makes it a dynamic stretch and as you continue to breathe and rotate, the torso will drop more and more easier.

After rotating around the foot about three to five times stopping around by the patient's little toc, he should pull the stomach in, tuck his buttocks in, return to the torso to the side bend position, then stand up straight. Repeat as needed on the same side and also to the opposite side.

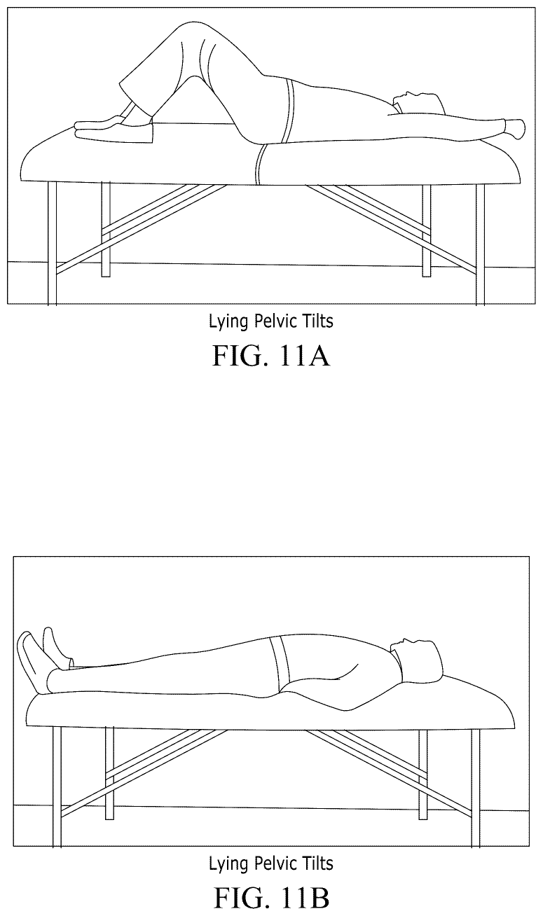

FIGS. 11A-B depict the lying pelvic tilt exercise, according to various embodiments. This exercise is helps develop stomach strength and builds endurance. It also provides a lower back stretch, so patients may notice their lower back feeling better from the lying pelvic tilt. But it is more beneficial for building strength and endurance for the abdominal muscles. There are two main variations of the lying pelvic tilt.

The first variation of the lying pelvic tilt exercise should be used if patients are fairly weak and have trouble doing the movement of this exercise. To do this variation the patient lies down with his knees bent and his arms down by his side. Or if the patient has a tendency to use his arms—which a lot of patients do—the patient can even keep his hands raised up by his head, rather than under it, to avoid pulling on their head during the exercise. While getting into this initial position, the patient will need to keep their stomach pulled in while they lie down. Otherwise, they will get a jab of pain because they typically will arch their back as they lie down. Having the knees bent prevents them from arching their back, keeping their back flat.

To proceed the patient should lie there, take a deep breath, and then push their lower back down to the bed to the point where they feel the pelvis tilt. Most patients will try to use their thighs to push down, so the doctor should monitor for this and point out the error in performing this exercise. Also, some patients tend to use their quads to push down rather than using the abdominal muscles. Again, the doctor should watch their quads to make sure they're not contracting.

The doctor should be able to see the patient's pelvis tilt as they do it, and then tell them to hold it for a count of five. Then let it relax. Doctor and patient can probably see the pelvis moving while performing the exercise. Repeat the lying pelvic tilt exercise, breathing deeply and exhaling. The patient should contract the abs, letting the leg muscles fully relax, if possible. Make sure they keep breathing, because a lot of people will hold their breath and that can give them a headache. So other than the patient's abdomen contracting, the patient's arms, legs and shoulders, and everything else, should be relaxed. Some patients may push so hard that they'll give themselves a headache from contracting their neck with extreme effort. It is best for the doctor to advise the patient to relax throughout this exercise.

The next pelvic tilt is done with the patient's legs out straight. The doctor should caution the patient to keep their stomach sucked in before putting their legs out straight, otherwise it will likely cause the patient's back to arch possibly resulting in pain or injury. So the patient should keeping his stomach sucked up into the ribcage, with his arms, legs and neck fully relaxed, then just tighten his stomach. This will cause the pelvis to tilt. It should feel about like the patient is trying to balance all his pressure on his lower back. If the patient puts his hand under his lower back (at about belt level), he should be able to feel it flatten against the bed. The exercise continues—deep breath, flatten the back. The position should be held for approximately 10 seconds, making sure that the patient keeps breathing to avoid getting a headache.

With their hand positioned at their belt level, the patient should feel where they're pushing. Having the patient push down should allow them to feel their whole body weight being supported right there. If the patient is weak, he won't have the strength in the abdominals, and will likely try to incorporate every muscle of his body. Typically, the patient's head will come up, and legs will come up as well. The goal is to get them to relax those muscles during the lying pelvic tilt exercise. The patient's head shouldn't move. It shouldn't be tightening at all [except for the abs]. The position should be held for ten seconds, and then relax. Deep breath, push down into your hand again, and relax.

Once the doctor knows the patient is targeting the right place, he should have the patient bring their arms up and let them hang above their head so they're fully relaxed. Having the patient do this will cause them to feel their buttocks clenching. A key to properly doing the exercise is to relax. Most patients find this will reduce back pain fairly quickly because it is easier to actually have them strengthen their stomach muscles before their back muscles. It's easier for them, it's less painful and they actually progress faster. Once the stomach muscles are strengthened the patient can work more on strengthening the back. But it's best to make sure the abdominal muscles are stronger first.

It should be noted that when you tighten one muscle group, the opposing muscle group relaxes. This is what happens with the pelvic tilt. If the patient is contracting his abdominal muscles, his lower back muscles are going to shut off because the brain shuts them off, so it isn't just a stretch.

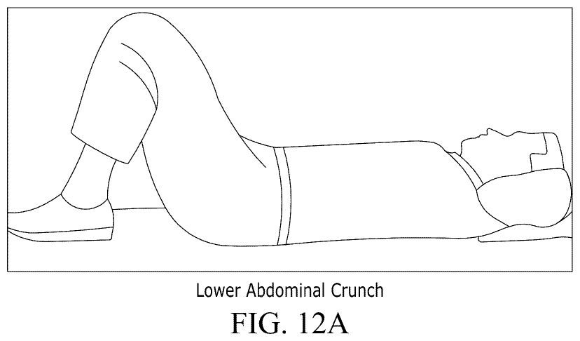

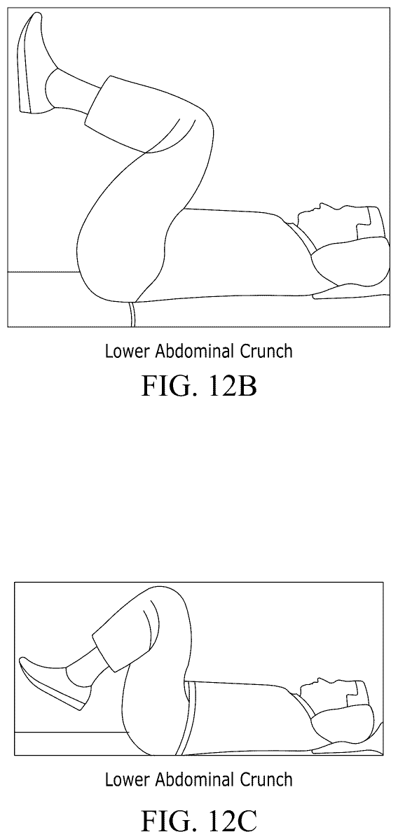

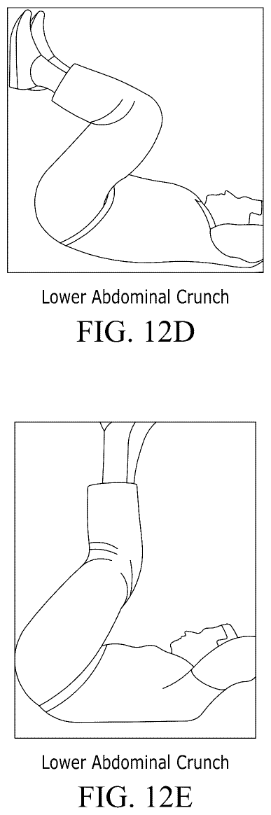

FIGS. 12A-E depict the lower abdominal crunch exercise, according to various embodiments. With knees bent, the patient lifts his knees up toward his chest taking care to avoid tightening his stomach muscles before lifting his legs. The patient then returns his feet to floor while keeping the stomach tight and lower back flat on floor. The patient should feel the squeeze in the stomach muscles.

The patent then lifts his hips and legs off floor by pushing his lower back into floor and tightening the stomach muscles. The patient again returns his feet to floor while keeping his stomach tight and lower back flat on floor. Finally, the patient squeezes his stomach muscles, pushing his legs upward toward the ceiling. The patient again returns his feet to floor while keeping his stomach tight and lower back flat on floor.

### Upper Body Stretches and Exercises

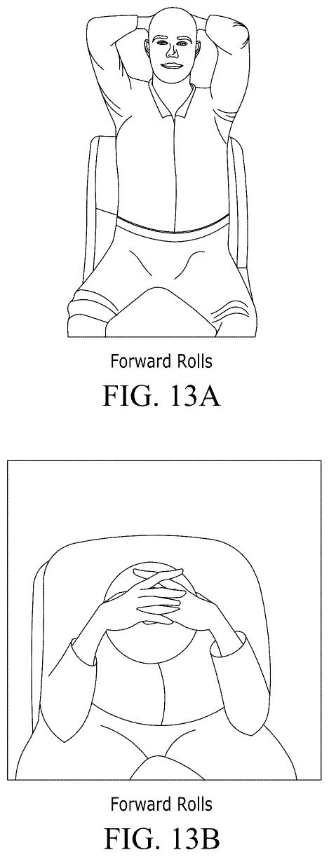

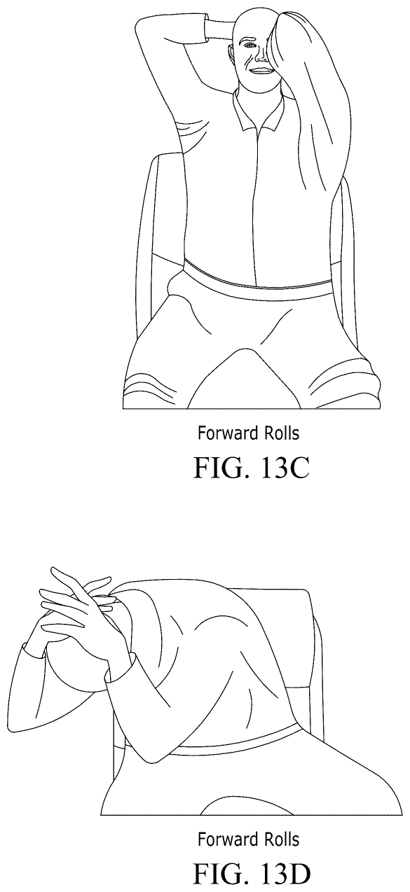

FIGS. 13A-D depict the forward roll exercise, according to various embodiments. Starting with the forward roll exercise is an effective way to warm up all the muscles. This exercise aids in increasing the blood flow as well as the general stretching benefits it provides, but also makes for a good shortcut for people who need to just very quickly hit certain muscles. It targets the lower back, thoracic, neck, and right up into the suboccipital, and actually has a stretching effect up into the occipital muscles as well.

While sitting upright, the patient interlocks his fingers, puts them at the top back of his head, and bring his elbows forward. The knees should be about a foot apart. Next the patient will retract his chin, keeping it retracted as he comes down one vertebrae at a time. That means the chin should come against the patient's throat to be in the retracted position, and not immediately down to his chest.

The patient continues to bend forward, vertebrae by vertebrae, all the while breathing and relaxing. The patient should feel a stretch in the neck, upper traps, and thoracic region, as he progresses down through the stretch, with his elbows moving toward his mid-thigh or about two thirds of the way toward his knees. Once the patient comes down, placing his elbows on his thighs, he will then reverse the motion and roll back on his buttocks, again sucking in the stomach and letting it lift the patient's torso. The patient should push up and out with his back as he rolls up, vertebrae by vertebrae, to feel a stretching sensation through the thoracic, upper traps and neck regions. During the roll the patient should keep his chin retracted, and bring the head and neck up to vertical, while remaining relaxed.

The same motion is repeated, except with the head bending down at about 45 degrees off center—i.e.. towards a knew instead of down the center. Again, the patient places his hands at the top back of the head, the elbows come together, with the chin retracted. The patient bends forward at an angle so that the left elbow is pointing to the outside of the right knee, while keeping the pelvis straight, but rotating the torso. Maintaining the chin retraction, the patient rolls downward, making sure to come down vertebrae by vertebrae.

Once the elbows have reached the thigh level, the patient reverses it and rolls back on his buttocks, rolling up vertebrae by vertebrae, again using the stomach muscles and pushing up and back into his thoracic and trap regions. In this case, the patient should really feel a stretching sensation in his left trap. The patient continues to come up vertebrae by vertebrae with the chin retracted until the head and neck are again fully erect. Some people let their head go back. However, it is preferable to stop at the vertical position. While this stretching motion is going on, the patient should concentrate on being relaxed.

One thing to keep in mind is that when the patient is doing the spinal roll, the doctor should view the patient from the side as well as the front or back angles. This will allow the doctor to keep an eye on the spine and see if any of the spine segments aren't moving properly. Some parts of the spine may remain flat even though they're flexing. So the doctor should make sure that the patient is pushing back, up and out through these parts of their back and neck into each vertebrae as they roll up. One way to assist the patient with that is for the doctor to put his hand on the part of the back that is staying flat, because they quite often can't actually sense that it's not moving properly. Whereas if the doctor—or even the patient—puts his hand there, the patient can push back into the hand and therefore have a true sense of what they're feeling.

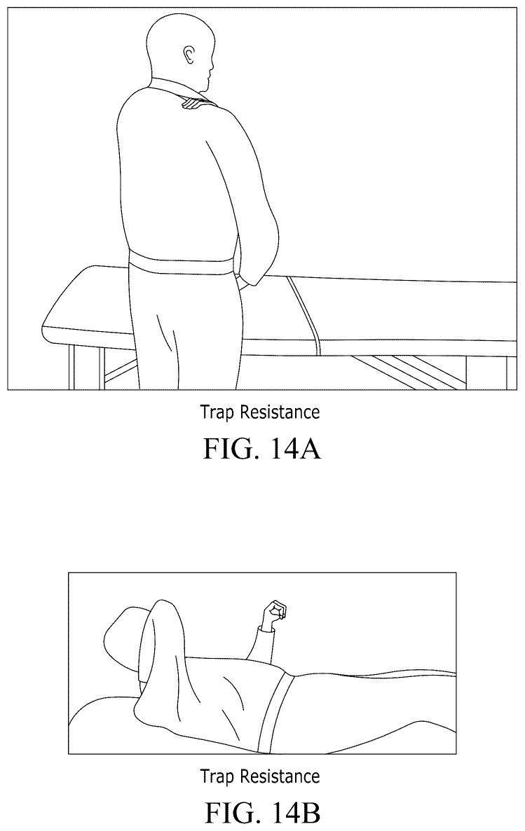

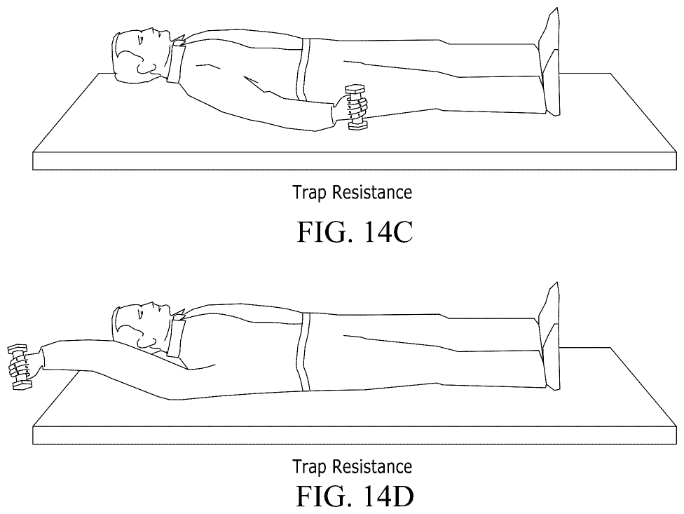

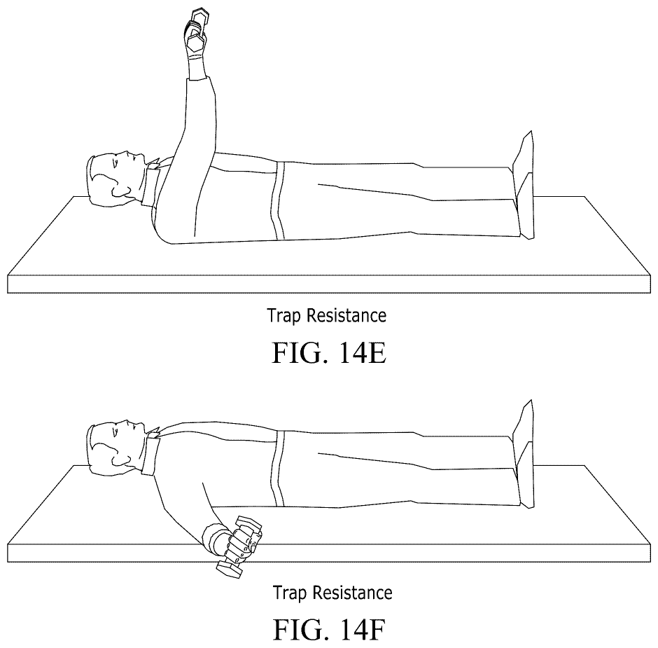

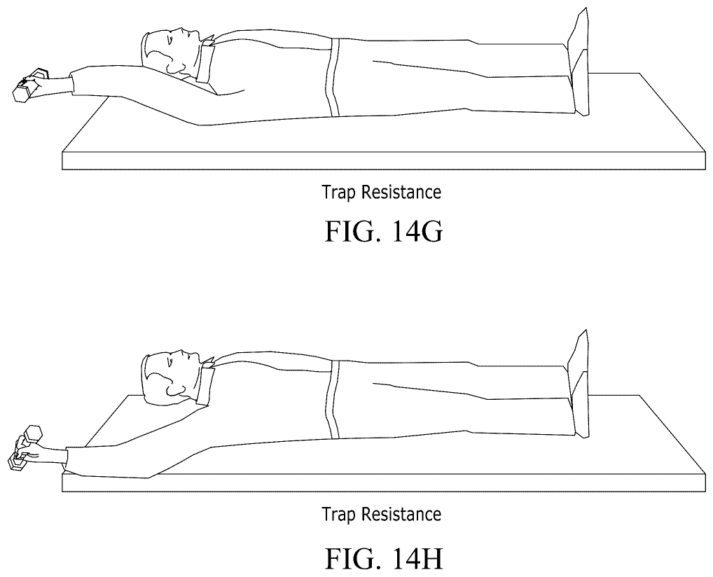

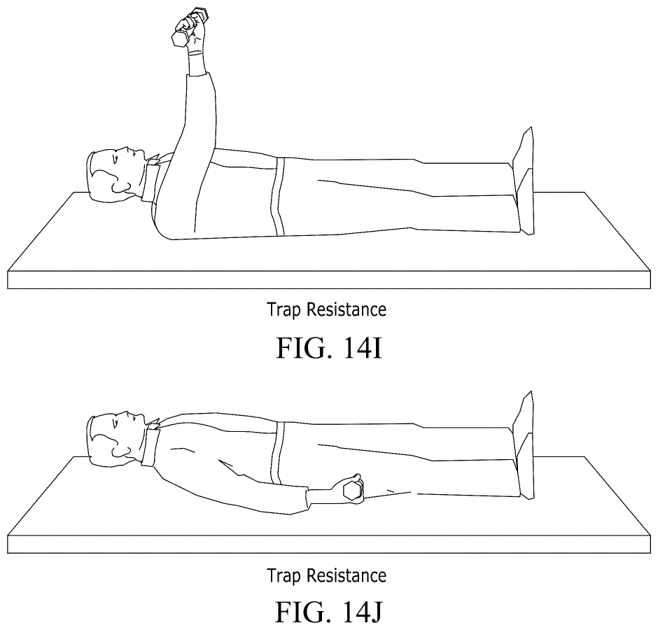

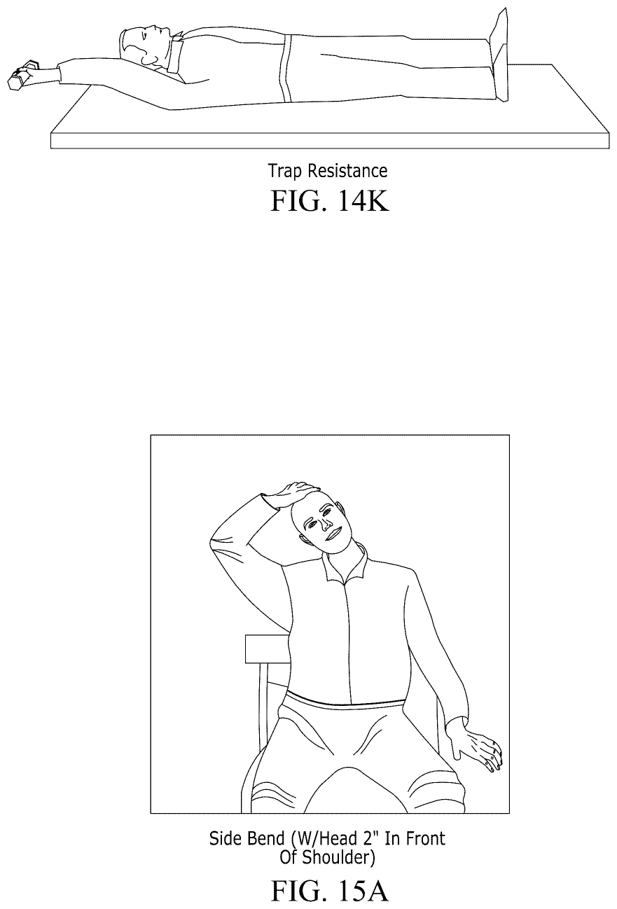

FIGS. 14A-K depict the trap resistance exercise, according to various embodiments. The trap resistance exercise is a combination exercise. The patient will typically lie on a bed or the floor to do this exercise. Preferably, the surface the patient lays down on is a bit firm, allowing the patient to feel it. To begin the trap resistance exercise the patient lies down on his back and feels the back of his shoulder for a little bump that everyone has to some degree, and that's where the trapezius goes over the brachial plexus. It's a bundle of nerves that really controls a lot of your upper body, so you want to make sure that you can get that stretched out, but if it's tight, you need to increase blood flow. To do this, we have to combine using the muscles to increase blood flow and stretching at the same time. A lacrosse ball can be used to help with this exercise by putting the ball under the little bump described above. Don't place the ball under the shoulder blade. It'll be very uncomfortable and you obviously don't want it on the spine, so it should ideally be positioned about halfway between the two.

In the hand of the arm/shoulder being stretched out, the patient can hold a light weight, typically two or three pounds to start. The weight is very light, so people find it quite easy, but effective. In this example, place the weight it in the right hand, the same side that the ball is placed (under the right trap), and bring the arm down to your side with the hand by your side. Then raise the arm back up the same angle keeping the elbow straight. And you raise it, you're using the muscles, causing an increase in blood flow.

But when the patient moves his arm back, he will feel the pressure of the lacrosse ball. Keeping the arm as straight as possible, bring it straight back then straight forward coming down as close to the bed or floor as you can. Repeat this motion five or six times. Then the patient extends the arm outward at an angle of 45 degrees from the hip, and raises the arm up to vertical. Orient the patient's arm turned such that the thumb is pointing up when he clenches the weight. Move the arm up at 45 degrees vertical and down, again five to six times. The patient should feel the lacrosse ball stretching the muscle where it's been placed. Now have the patient move his arm out to 90 degrees away from the torso, and raise the arm straight up to vertical and lower it straight back down again five to six times.

The next position is one most patients will really feel. Have the patient extend his arm to about 135 degrees away from your hip, so the arm is basically 45 degrees off the center line extending out of the top of the patient's head. Once again raise the arm upward, vertically and lower it all the way down. Be sure to keep the angle at 135 degrees. The patient should raise and lower his straightened arm with your thumb pointing up, five to six times.

The last motion of the arm is sometimes called the “snow angel” motion since it's sort of like half of a snow angel. With the palm facing up holding the weight, and the thumb extending towards the patient's head, have the patient raise his arm slightly, take it from his side and move it across up and above his head (like he would in making a snow angel). The patient should feel it change over different muscle fibers under the pressure of the lacrosse ball, and should feel how the muscles are at different angles when going through that motion. Again have the patient raise and lower his arm five to six times.

Then you repeat it on the other side. It's not a difficult exercise, but has an enormous effect on the traps. If the patient does this before doing the trapezius stretches, they will generally be much easier to perform. The progress will be much faster as well.

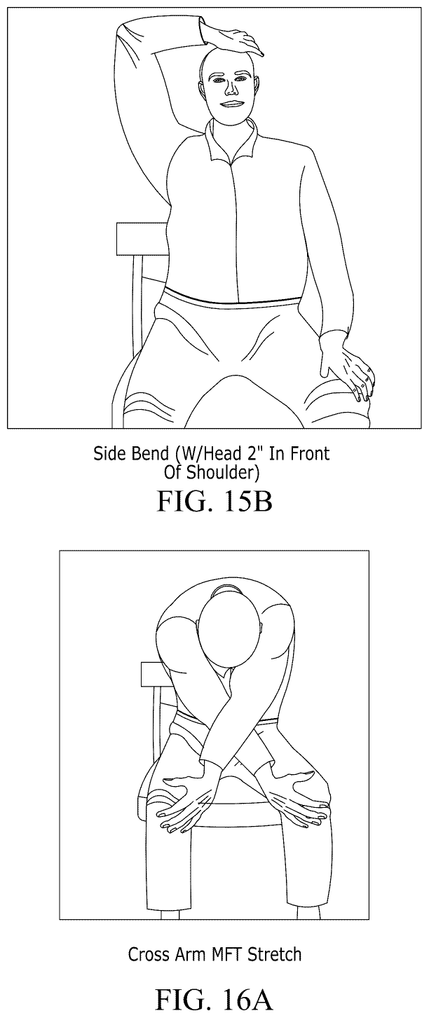

FIGS. 15A-B depict the side bend stretch (with head 2 inches in front of shoulder), according to various embodiments. Sometimes the conventional upper trap stretches will not be very effective if the upper trap is too tight, particularly across the top of the upper trap. In this muscle region, if it's too tight, when you try to stretch the medial scalene the trap will prevent getting a proper stretch of the medial scalene. Most people really don't differentiate and don't understand the difference of where the trap is and that it's behind the medial scalene.

This can be felt by starting to push one palm down, and as you're dropping the shoulder again retract the chin. Then bring the ear down toward the shoulder, but bring it down so it's two to three inches in front of the shoulder. You should feel a strong stretch right along the top ridge of the (upper) trap. Remember that breathing is essential. Maximize the breaths in and out. Keep the shoulder back (of arm with palm down) against the chair, and with every exhale, the head drops an inch or more. The shoulder has not left the chair. Then roll back up, keeping that palm down.

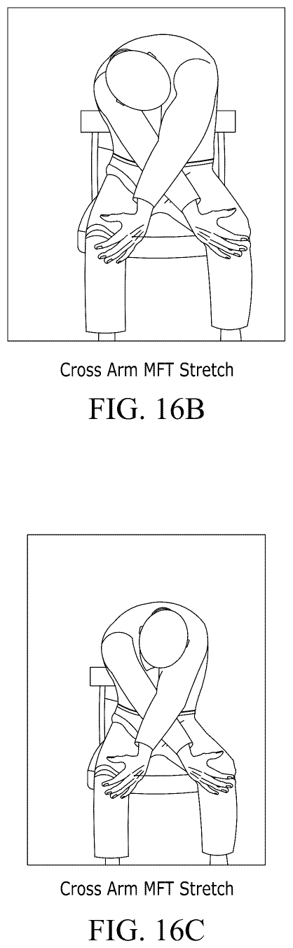

FIGS. 16A-C depict the cross arm myofascial tension (MFT) stretch, according to various embodiments. This stretch actually affects the mid trap arca, but may also get some of the lower trap. For this reason there isn't a need to stretch the lower trap separately. This stretch provides a bit of a combination of stretching the two. The cross arm MFT stretch starts by crossing the arms and grabbing the legs maybe two or three inches below the knee—that is, grabbing at basically the top of the calf. The patient should take a deep breath and push up between his shoulder blades as he exhales. The patient leans to one side, then to the other side while breathing deeply.

You may feel this stretch actually pulling all the way down into the lower back. It is interesting that patients often suddenly start to recognize from that they're stretching both their mid and lower traps. But they also feel stretching in the lower back. So it gives a good example of how it's really a continuous chain of muscles that pull from the neck right to the base of the spine. For this reason it is a very good learning tool for people too.

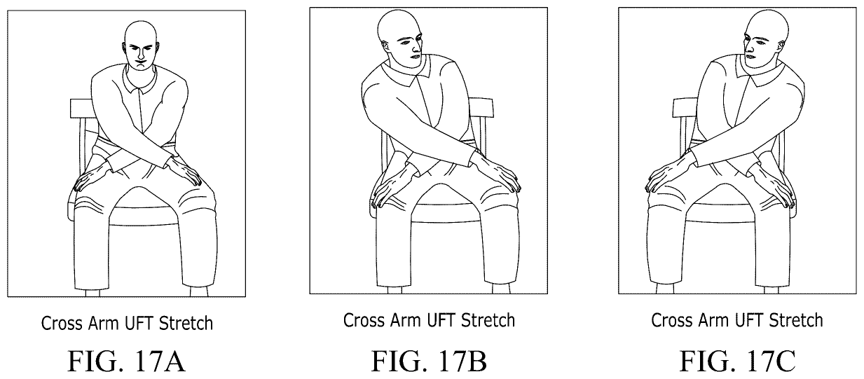

FIGS. 17A-C depict the crossed arm upper fiber trap (UFT) stretch, according to various embodiments. This is a very simple upper trap stretch. It is important with this stretch to exercise the middle of the upper trap as well as the sides of the upper trap. The patient crosses his arms and grabs the outsides of his thighs just above the knees. Then the patient drops his head forward to get the upper attachment of the traps and pushes up along the base of the neck/top of shoulders while breathing deeply (about 3 times) with maximal inhalation and maximal exhalation to get a full and proper stretch in the upper traps.

The patient then leans back with the torso at 45 degrees away from the thigh so that the top arm is grasping the side to be stretched. This should be performed three times on each side, and maybe twice in the middle. Note that most of the time when patients aren't stretching well, it's because they're not doing maximal inhalation and maximal exhalation of their breathing.

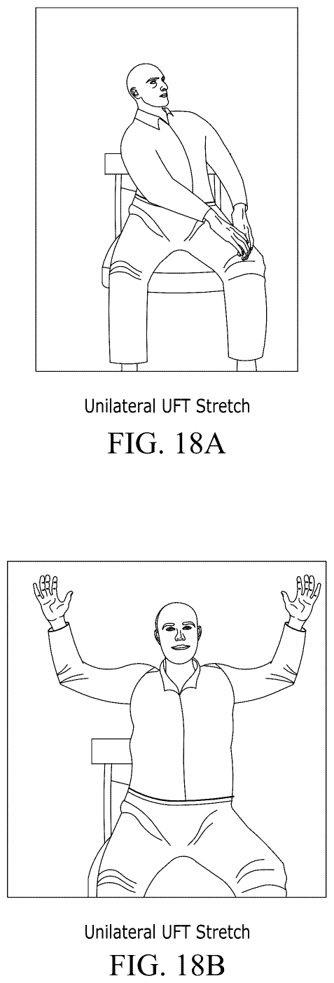

FIGS. 18A-B depict the unilateral upper fiber trap (UFT) stretch, according to various embodiments. In this stretch the upper trap will be stretched using a single arm (i.e., just on one side). The patient sits forward about three inches from the back of the chair to have enough room to do the stretch.

The unilateral UFT stretch starts with isometric contraction. The arms are moved up with the elbows shaped like a big ‘Y’. The patient squeezes his shoulder blades together, allowing them to feel the stretch mainly in the upper trap. Then the patient moves his arm downward, in this case their right hand is going to the position outside of the left knee. With the left hand down on top of the right hand, the torso is rotated. The patient takes a maximal breath in, and keeping his chest out, the patient brings the shoulder back and down because you have to a specific point where the brachial plexus is located. The figure depicts how the patient stretched a lot, but with his shoulder still back and down.

The patient remains flat across the back of the shoulders. The patient will often feel it pull into the shoulder joint, more the anterior delt in fact, because the nerve from the brachial plexus actually goes to the anterior delt. So it may cause pain in the joint as they do it, but the second time they do it, it will have stretched out, resulting in little or no pain.

There is one point that makes this stretch a little bit better. It again stretches the upper part of the upper trap, the same as the one as above where the patient stretched the ear two inches in front of the shoulder. In this stretch when the patient is leaning back, he drops his head away from the side being stretched. This should make the patient feel the stretch across the top of the upper trap. It is a good habit to get people into because any reinforcement of that is a positive outcome.

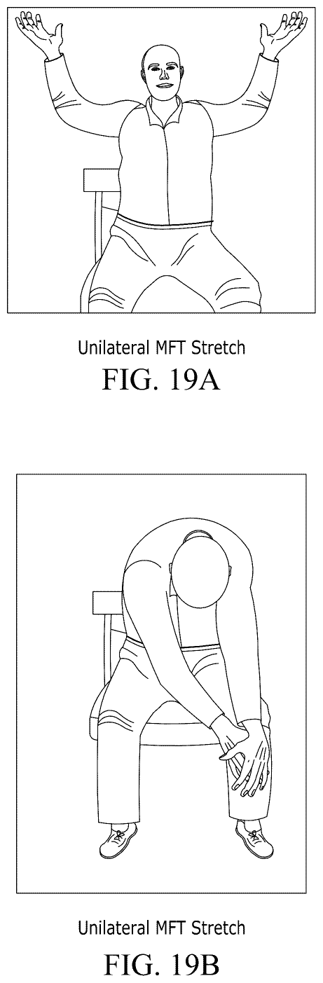

FIGS. 19A-B depict the unilateral myofascial tension (MFT) stretch, according to various embodiments. This is another mid-trap stretch. The cross arm MFT stretch described above worked the sides of the mid trap a bit, but it is aimed more in the center of the mid-trap. The unilateral MFT stretch is going to be used to ensure to distinctively stretch the sides of the mid-trap. The stretch begins by bringing the patient's arms up like a big “T” again. When the patient squeezes his shoulder blades together, he should feel the stretch between the shoulder blades. This is the mid-trap.

The patient then leans forward and uses his hand to grab the mid-calf on the opposite side of body, and put the other hand on top of it. The patient curls his back up like a cat and then takes a deep breath, blowing it out as he pushes up his back through that mid-trap area. This should be done for three deep breaths in and out.

The patient should now relax. The stretch is held for approximately 15 seconds. That's really adequate for that—enough so the patient should feel a strong stretch there. The stretch can be compared it to a cat-stretch in the back, which is something most people can relate to as far as the movement itself.

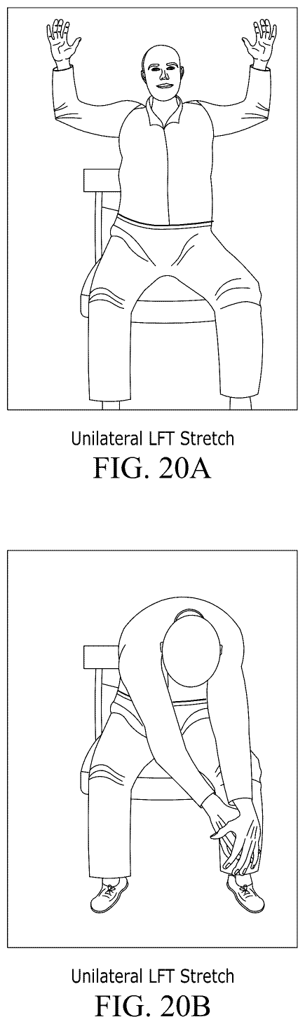

FIGS. 20A-B depict the unilateral lateral hip flexor tension (LFT stretch, according to various embodiments. This is a lower trap stretch for one side of the body. The stretch begins by bringing the elbows back so the position of the patient's upper arms looks like a “W.” The patient brings his shoulder blades together tightly, then let his arms down and leans forward with his hands reaching down to the outside of the opposite side ankle. Some patients may grab the bottom of their opposite side foot. Other patients don't have arms long enough to do that. It all depends on the length of the person's torso and their arms. The patient then grabs the outside of the ankle and pushes up through their back just below the shoulder blades. This is done while inhaling and exhaling for three deep breaths while pushing up the back but still holding the ankle (or foot). Complete the stretch by rolling back up.

It may be best to avoid this stretch on the right after having eaten a large meal. It can be painful bending on the stomach, doing this exercise. This should be taken into account if the patient has just eaten or had a snack. The unilateral LFT stretch can be compared to a cat-stretch in your back, with the patient pushing up below the shoulder blade more and more on each successive breath.

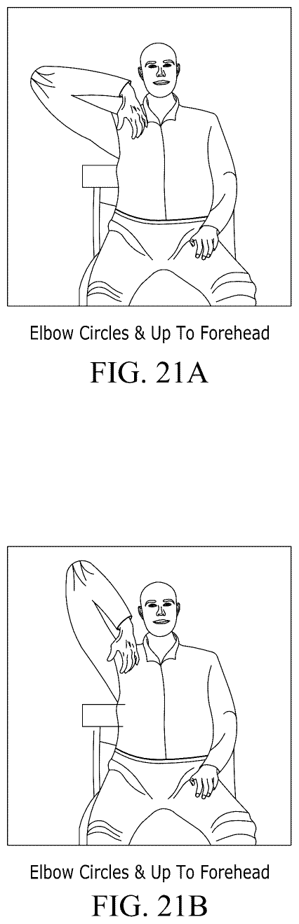

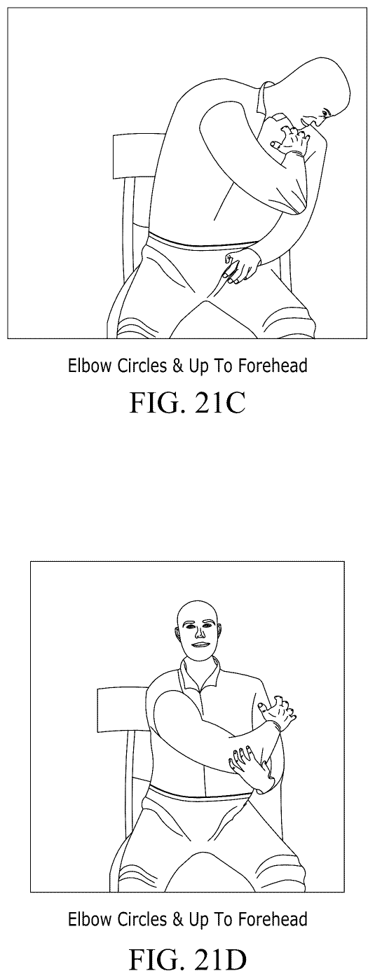

FIGS. 21A-D depict the elbow circles and elbow up to forehead stretch, according to various embodiments. This section will include two other exercises worth keeping in mind in case patients aren't getting a good enough stretch and especially if the patient experiences tightness in between the shoulder blades and mid-trap area. The first stretch is a simple elbow circle, which some patients seem to have tremendous trouble doing.

The patient first brings the arm up, then lets his hand hang in front of his shoulder, pointing his elbow upward. The patient raises his elbow upward as high as he can raise it towards the ceiling. The patient brings the elbow back across his front to the outside of his opposite knee, pointing it downward at about a 40 to 60 degree angle, depending on where the tightness begins. The patient pushes the elbow down while curling the back up.

The patient stretches the elbow in opposite directions in this stretch, which generally results in a strong stretch, and then continues to swoop on back, up to the ceiling, over, pushing down with the elbow, and pushing up in the shoulder blade. This often results in a tremendous amount of relief for the patient. They may even feeling relief if there is pain along their sternum, which is the referred pain from the mid thoracic area putting pressure on the intercostal nerves that go to the sternum.

The second stretch involves similar movement. The patient brings his bent elbow up, using the other hand to pull the elbow across the body. The patient then uses the other hand to push his bent elbow up to his forehead, and then with the hand pulls the elbow down across the chest. This motion can be done three times.

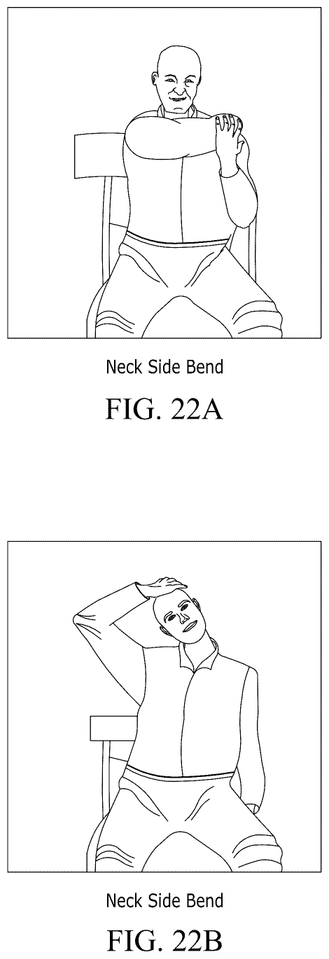

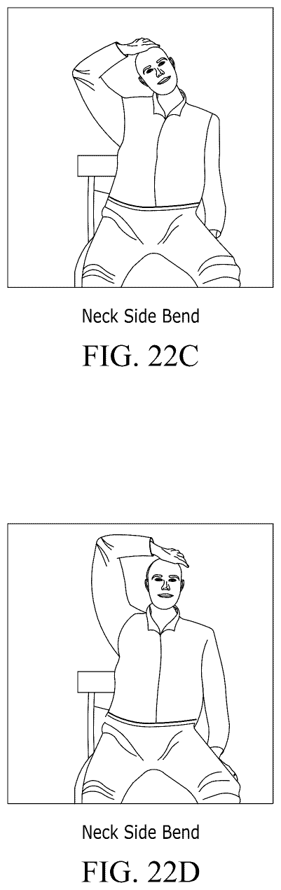

FIGS. 22A-D depict the neck side bend stretch, according to various embodiments. Patients with pain down their biceps or in their forearm is fairly common. Conventional diagnoses tell them that it's due to tendonitis. This, technically, is true. But it's actually caused by muscle up in the neck and shoulder regions putting pressure on a motor nerve, causing muscle in the arm to contract. Another example is patients with pain in the thumb. That may be due to pressure on the median nerve. For these conditions we need to address the scalene muscles. In particular, for these cases, the medial scalene needs to be addressed.

The patient begins the stretch with one hand up, keeping the chin retracted. Most patients will bring the head forward and end up stretching the wrong muscle. They get the anterior scalene or posterior, but they seldom, if ever, stretch the medial scalene. The patent positions his hand over his head with fingers above the ear, chin retracted, while pushing the opposite palm down. The scalene attaches onto the vertebrae but it also attaches down onto the first rib, so the patient has to be dropping the shoulder to stretch the full scalene. The patient then takes a deep breath, keeping his chin retracted, and puts the ear down to the shoulder, exhaling. This is repeated for another inhalation and exhalation, usually for three or more deep breaths in and out.

The patient keeps pushing down the palm and dropping the same side shoulder and then rolls his neck to the side, pushing his neck outward to the side. A variation the present inventor includes in this is to stop about half-way to the neck being straight, with the patient's skull tilted relative to his neck. This motion may well stretch the scalene and the RCL (Rectus capitis lateralis) at the same time. The patient takes another deep breath, and without bending his neck any more, tilts the skull a bit further. The patient then rolls his neck and pushes his ear up to the ceiling as he brings his skull and neck straight upright. The neck side bend stretch is really getting at two different muscles at the same time. This alleviates multiple problems because if the patient has trouble with the RCL, he will also have problems relating to the facial nerve or trigeminal nerve, and problems with the medial scalene will relate to problems with the median nerve.

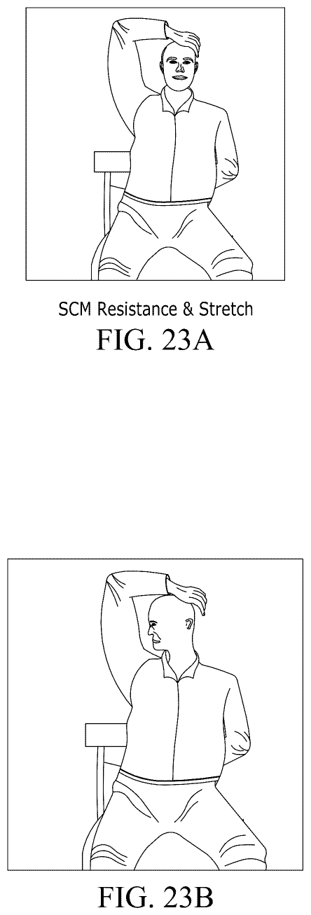

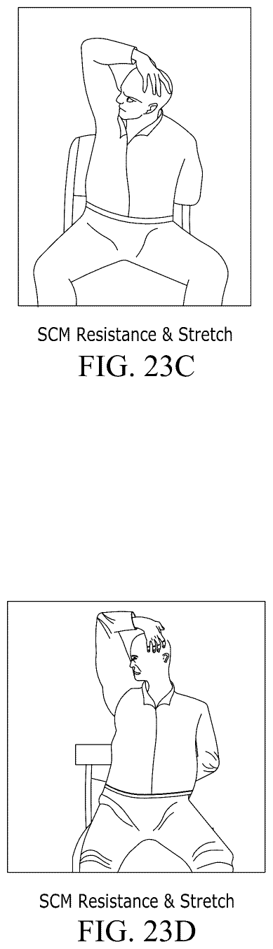

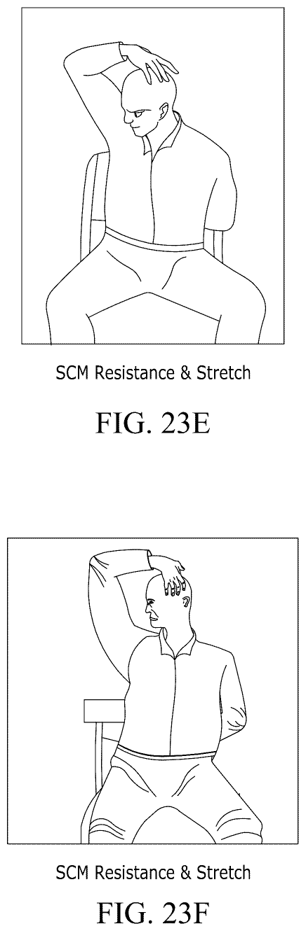

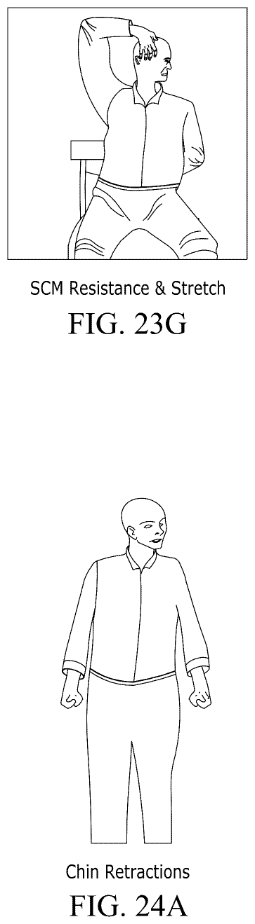

FIGS. 23A-G depict the sternocleidomastoid (SCM) muscle exercise and stretch for treating physical disorders, according to various embodiments. The previous exercise involved stretching the ear to shoulder, a stretch included as the first of a series of exercises for the SCM or sternocleidomastoid. The patient won't be able to rotate his neck if the scalene muscles are too tight. But the next logical stretch goes into the rest of it, which is to bring the hand up again, just the same as last stretch, but this time the patient is going to put his hand behind his back (the other hand). The patient should avoid tilting his skull, and instead just rotate it, so as to look towards his armpit. The patient takes in a maximum breath, and then moves his left ear down to his left knee by pushing the side of his head. It is important to push on the side of the head, and not the front. With the front of the head people tend to tilt their skulls forward so the nose is down. To avoid this the side of the ear should move down towards the knee (your side of the head coming down the knee).

Upon taking a deep breath, and while keeping his right shoulder back, the patient moves his head forward, pushing and hold it for about three seconds. The patient should push fairly hard with his fingertips, then abruptly let go. That causes a surge of blood flow and the muscle relaxes more. This should be done three times to each knee.

Now we're going to, again, head upright, bring the elbow back, and you have to make sure [the elbow comes back], people tend to keep their elbow just straight out and they try to rotate their head, which you can't go very far. You have to make sure they take a deep breath, exhale, bring the elbow back, and then bring the ear down, your left ear, to your right knee. Push hard into the fingertips for three seconds, then let go.

Now you'll feel the muscle start to let go. And the SCM, everyone deals with the SCM in varying ways, but the only part that really matters for SCM symptoms is actually within one inch here of the attachment to the mastoid process. So that is a part that should release if you're going to alleviate vagal symptoms.

Now, this time again, the head is upright, we've just done the resistance, which increases blood flow so now we need to stretch it. So again, fingers over top of the ear, but actually put them a little bit in front of the ear, so almost [at the] temple. Take a deep breath, bring the elbow back, rotate the head as far as you can and as you can see, I can use my fingers to rotate a little bit further. Deep breath, exhale, take another deep breath. I slide my fingers back towards the back of my head and I can rotate the opposite direction. And so all you're doing is sliding your fingers back and forth along the side of the head. So it's a very simple movement once people understand it, you're using leverage. But the shoulder, the other shoulder, stays back the whole time. You don't let it round or the body's rotating, the muscle isn't stretching. So I do that three times and I finish with the stretch and then I relax.

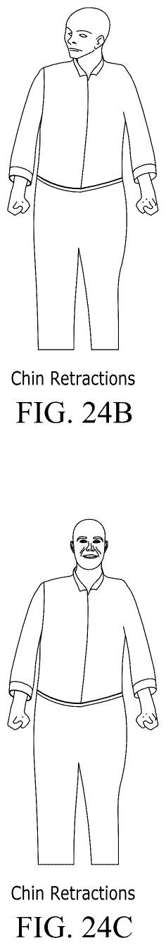

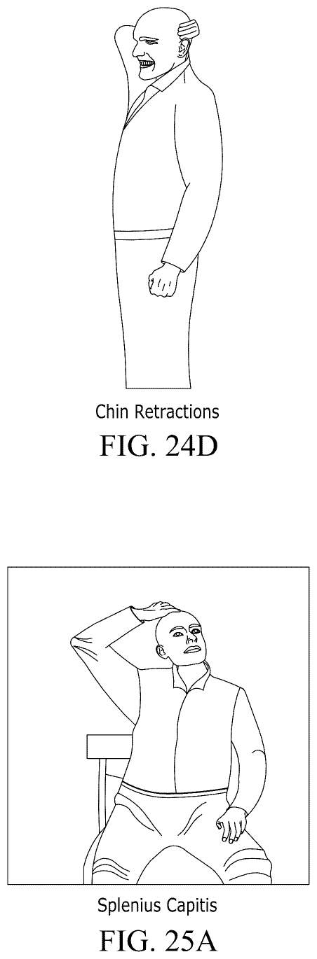

FIGS. 24A-D depict the chin retractions stretch, according to various embodiments. The next exercise you're going to do is called a chin tuck, which sounds very simple, but to do it properly is a little bit more complex. So you stand with your feet about six inches out from the wall. Your back against the wall, your shoulders back against the wall, so they're going to be back and down, and the back of your head against the wall. Now because the traps go up the back of the neck, on either side at the back, and it's a very large muscle, so often it's easier to start to get this stretch by starting with a 45 [degree] rather than pushing straight back.

I'm going to rotate my head 45 degrees, back of the head against the wall. I'm going to just tuck my chin, which is going to flatten my neck against the wall. And what it feels like, is like I'm sliding the back of my head up the wall and I let it relax. So I slide the back of my head up, so it makes me feel like I'm getting taller and I can feel that pulling all the way down my neck, the back of my traps, the back of my shoulder, and right down into the shoulder blades and actually even down to my lower back when I do this. You hold it for about five seconds, then relax. The chin comes back to normal. Try it one more time.

Chin goes back, head goes up and I don't feel pulling as far down my back now, and I relax. Now you're going to do exactly the same on the right side. I'll only do it once, so you're going to rotate 45 degrees, slide the back of the head up the wall so I'm flattening my neck against the wall. Hold it for five seconds. Relax. Now, since I've stretched both sides, now it's going to be easier to hit both sides at the same time. So I'm just going to retract my chin, slide my head up the wall, hold it for five seconds and let it relax.

Now what you might, and I don't think you can see it from this video because I'm doing it from the front, but if I turn sideways now this is my normal stance, you'll see that my ear is much more over my shoulder than it would've been actually earlier, because it has loosened the muscles at the base of the skull and up the back of the neck, which allows your head to come back or basically retract your head, which is bringing your chin backwards like a chicken. So just to give you an example, I'm going to pretend that my hand is the wall so you can see the position of my ears, which, because I've already done it, they're reasonably good. But you can see how my neck is flattening when I push back against the wall. Now let it relax, and your head tends to stay in that position after that.

Flattening the neck against the wall generally feels very good. It will help headaches at the forehead, but it actually allows you to and take pressure out of your forehead and out of your head, the front and back and top. But it's a good lead-in to the next exercise.

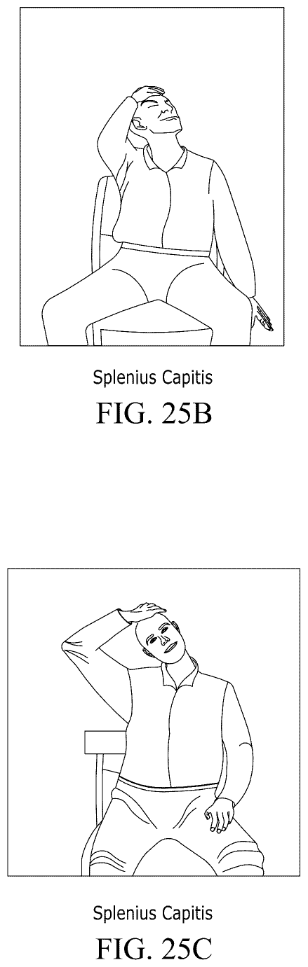

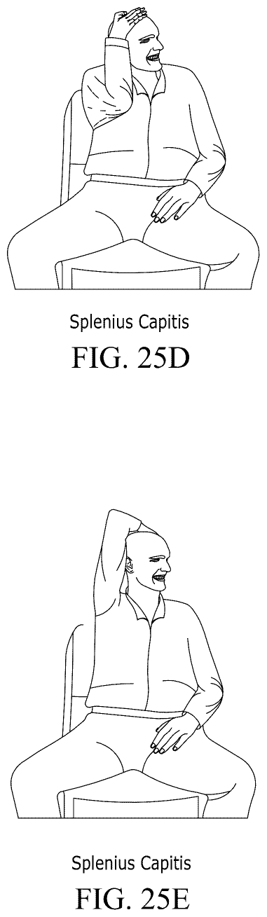

FIGS. 25A-E depict the splenius capitis stretch, according to various embodiments. Now an extremely important muscle in the neck to address is the splenius muscle. Now the splenius is very poorly understood by people because of its rather different angles that are involved with its structure. So we should stretch it out, otherwise you're not going to do a good job with the upper trap, with the 20-40-60. A lot of these exercises you're not going to do correctly if you do not get the splenius.

What we do now is again, fully upright, you keep your chin retracted. You bring your hand up, here above your ear. Take a deep breath, exhale, bring the ear down to the shoulder. Deep breath. I'm going to rotate my head forward, and you can see it's not going down very far. I want to see if I can get my head down to being parallel with the floor, it's a long way from there right now. So I rotate back and forth and I find a point, like right there for me. You probably saw my head pop up a little bit, so it's very tight there, so I might stop and take an extra breath or two. Then I continue with the rotation. And you see how much easier it is for me to come lower. Now I want to keep my shoulders back, keep the chin in, and I'm just coming forward to about 80 degrees (my head is 80 degrees towards the camera). I come to about 80 degrees and I'm going to bring my left ear behind my left shoulder. So I'm going to press the side of my head into my fingertips as I go back over my shoulder and relax. And you can do it again.

Ear to shoulder, rotate, but you see I'm starting much lower this time. There's really no point there where I have to stop and breathe. Then I bring it back over my shoulder. Now one thing I'd like [you] to know is sometimes if people's traps are too tight, they can't bring their head back over their shoulder because the trap will go into spasm and they'll get a very sharp pain in the upper trap. So they can still do it, but they only come until upright, or even if it's not fully upright, that's fine too, [they go] as far as they can without triggering the trap spasm. It means you're probably going to have to go back and do one or two more upper trap stretches. Then they'll be able to bring their head back. But don't worry if they can't go all the way, just have them continue trying it.

One other refinement you can add, and it's a wise choice with the splenius exercise, is with the arm on the side that's down, away from the side you're stretching. You push the palm down so it keeps the shoulder down because when the person rotates the head, they're going to feel a stronger stretch of the splenius if the shoulder is dropped. So you'll get that much more efficacy from that exercise. So I strongly recommend you ask them to push their palm down while they do it.

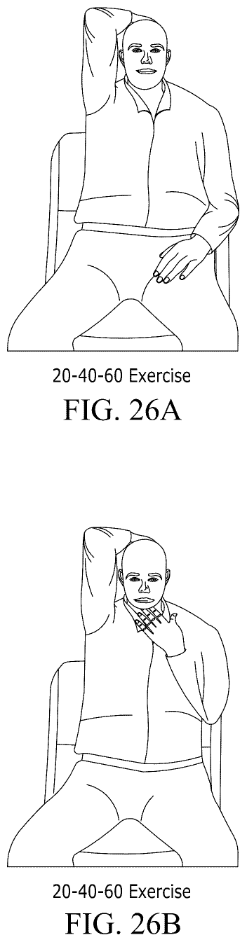

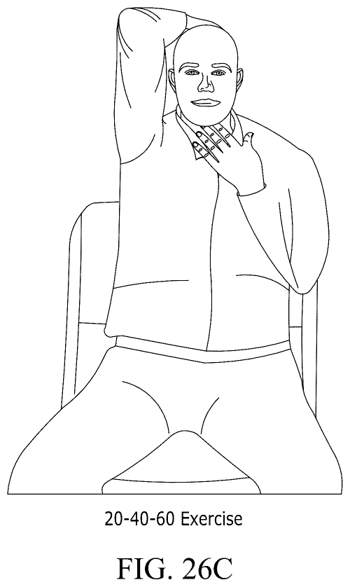

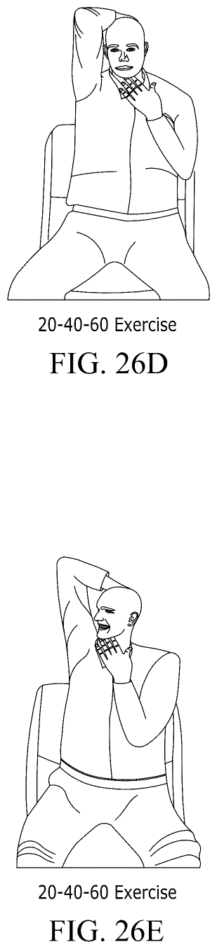

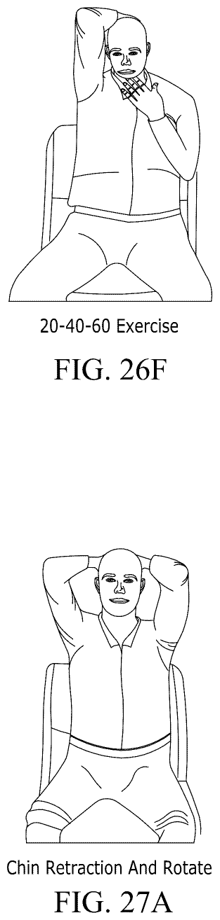

FIGS. 26A-F depict the 20-40-60 exercise, according to various embodiments. The next sequence of exercises is extremely important in many, many ways, because typically, as we talked earlier, our traps are tight, which cause our shoulders to round, and it affects the muscles up the back of the neck and the trap goes on top of those neck muscles. So you need to stretch both. So obviously you have to get it from a variety of angles or you're not going to get all of it, because they tighten all across the base of the skull.

What we first start with is you bring your hand up, you want your forearm in line with your head, grab the back of your skull and you can see my elbow is sort of in front of my nose. I'm only rotated about 20 degrees. I keep my chin retracted, I take a deep breath, I don't let the chin move. It can go back, but it doesn't come down to my chest. You can see I'm really rotating my skull around my chin. So I'm feeling a strong pull up the back of my neck down into my shoulders, and up the back of my skull actually. Now I'm going to roll up. So I'm going to start rolling and I'm going to push the base of my skull up to the ceiling, like I'm trying to make myself taller, like a giraffe. You almost feel like you're pulling yourself out of the seat, then when you get to vertical, you stop and let it relax.

Now something that makes it a lot easier, which you saw me do there, I put my thumb underneath my chin and against my forefinger in front of my chin. So it helps me control that my chin doesn't come down to my chest. It keeps the chin retraction in place. Because when people first start, they tend to bring their chin forward and down to their chest, instead of keeping it up, which means you'll get the lower neck, you will not get the upper three vertebrae or up into the suboccipitals. So any of the symptoms that come from that area, of which there are many, you will not touch at all. So this is very important. Thumb under the chin, forefinger in front so it can't move.

Now do the 40 degrees. Deep breath in, keep my chin retracted. Exhale. . . Roll up, now push up towards the ceiling until you're vertical, then stop. Now, one thing I want to remind people, is that doesn't mean that when you're coming up, first of all people let their chin slide so it comes down, but you want to keep it retracted. But when you're coming down you're fine, but I don't want people coming up until the head comes back like this because then you're contracting the muscles again. That's not the point. You're going to get the stretch and the activation by the roll itself and the stretch should be while you're coming down. And obviously it's going to be exactly the same as sixty degrees, I'll show it just briefly. So I'm going to get my chin retraction, rotate, and this is the hardest. Roll, push up and just come to vertical.

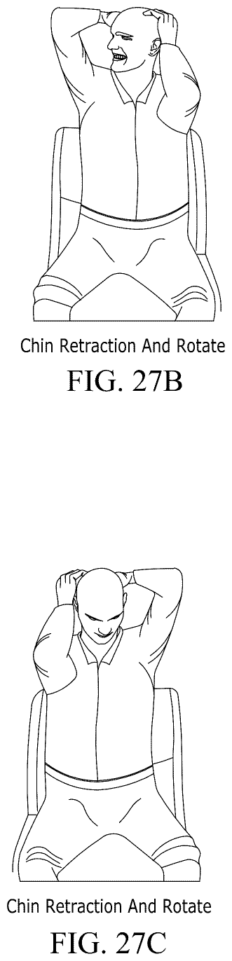

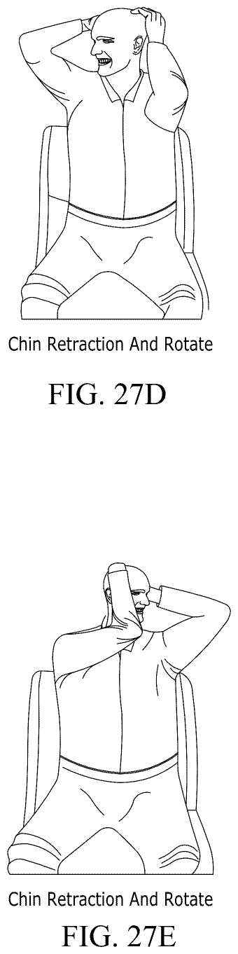

FIGS. 27A-E depict the chin retraction and rotate stretch, according to various embodiments. Now an excellent way to, or the next best exercise after 20, 40, 60 is you interlock your fingers, retract your chin, hands top back of the head. And I don't mean the back of the head down here cause that'll just push the head forward and you only get the lower neck and you can't maintain a retraction. Has to be the top back. So I retract. I bring my elbows together. I roll my skull forward. Then I rotate side to side but you'll see my chin is not coming down to my chest. But in fact, I'm able to retract my chin further as I do it, although it's pulling down my back. And you try to go the same distance each side. You can see that I'm tighter on the left. As I'm going to the left you see, it's much more difficult for me as it typically is for a right dominant person. Now, the key here is to keep the chin retraction. And by the way, if you've done your 20, 40, 60, and then you do this chin retraction and rotate. Sometimes you'll find a specific arca, that's tighter and you can actually come up at that specific angle because you may have missed the exact point, maybe the tightest point wasn't at 20 or 40 or 60 but could be anywhere between. This lets you check it out, see where you feel it and then you can roll up at that location. Now, the other chin, retraction rotation is actually different. This time, you move your neck and your head. You're not just rotating your head. So again, interlock fingers, top back of head, chin retraction. I come down. This time, I'm rotating my head and neck. It's a different plane. So you feel it very differently. So it makes a nice way to end off the exercises.

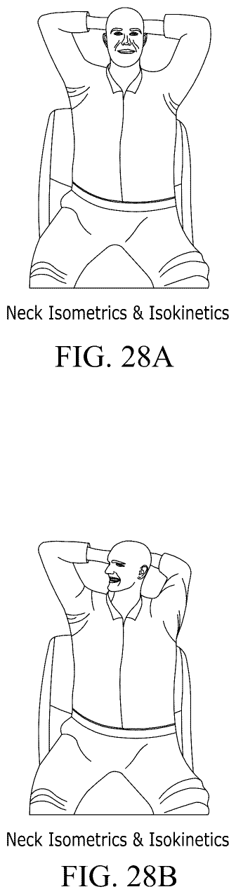

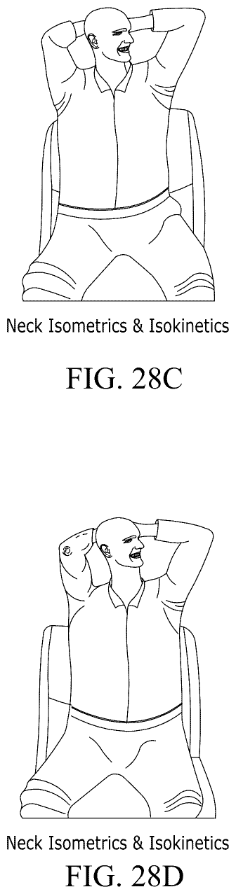

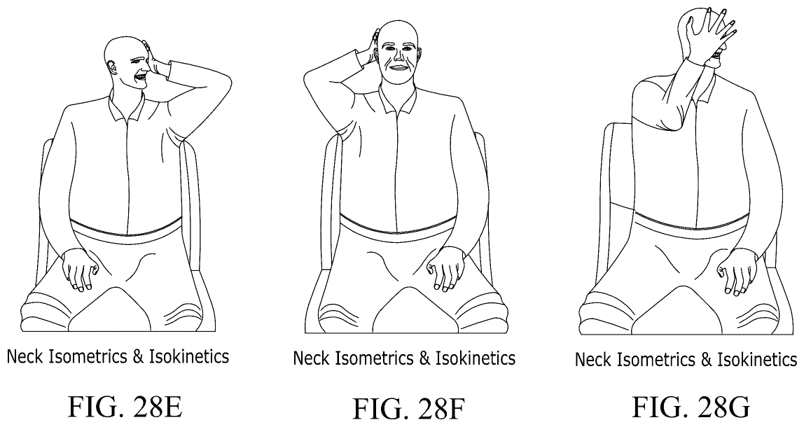

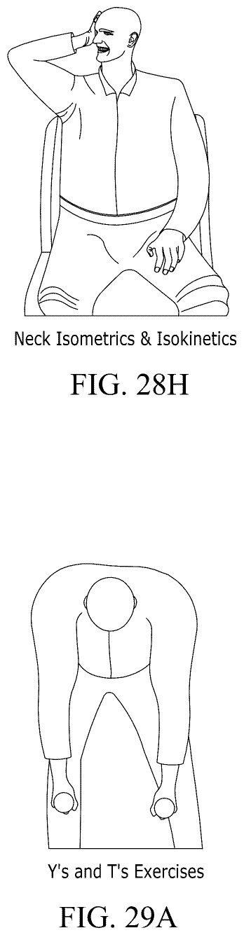

FIGS. 28A-H depict the neck isometrics and isokinetics stretches, according to various embodiments. What about if you feel tight afterwards, or you feel discomfort from the stretching, people are like “oh, I stretched too hard”. And even though you warn people not to, on occasion, they will stretch too hard. That's just human nature and we often have those overachievers, in fact, a lot of them because they're the ones who are the worst to try and keep on track, they always want to be better yesterday. So what we want to do is give them relief. Show that even if you over stretch a bit, you can give yourself almost instant relief.

So, say I'm feeling it in the back of my neck from what I just did with the chin retraction. Again, fingers interlocked, my head is upright, I retract my chin, and I just push gently back into my hands for about 3 seconds. Let it relax. Again, three seconds. Let it relax. You see I'm not straining. It's just to activate the muscles to increase blood flow. I'll try at 45 (degrees). And relax. Relax. And I'll do 45 degrees, then relax. And now my neck moves very freely and I don't feel any of the stretches discomfort, it can be gone.

Now of course if a person has a slightly different angle where they've over stretched, you'd go to that specific angle to exert the pressure back. But that can be the SCM too because let's face it, sometimes people just go too hard with their rotations and can overstretch. Which probably isn't the healthiest thing. So, instead we put our palm against our cheekbone. And it should be against the cheekbone. It shouldn't be on the check. It shouldn't be on the temple. It's on the check bone. You retract your chin, elbow stays straight out to the side. Gentle resistance and rotate. Bring your elbow back. Gentle resistance. Go all the way around the other side. Again gentle resistance to the front. Let it relax. Same thing to the other side. Gentle resistance. All the way around the other side. And just come to the front and relax. And that should leave all the neck muscles feeling quite good or at least then you've isolated what is left and you can repeat what is appropriate.

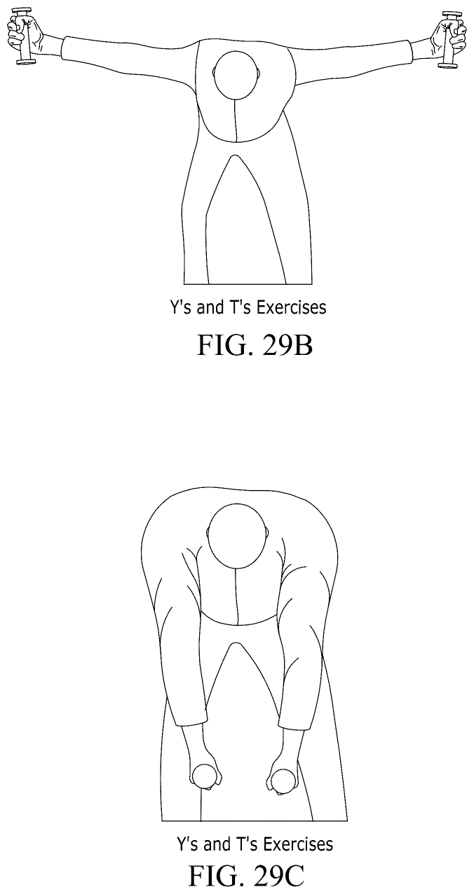

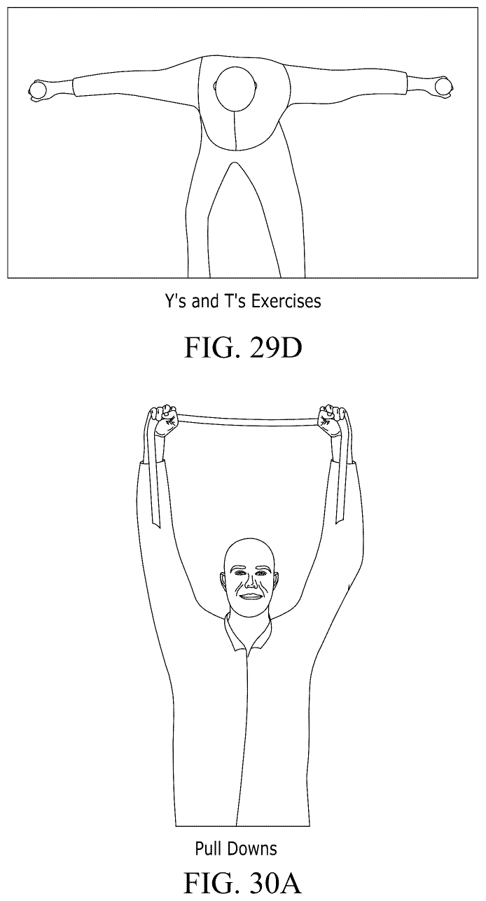

FIGS. 29A-D depict the Y's and T's exercises, according to various embodiments. We're now going to do the Trap strength/endurance building and realistically endurance is more important than strength. Strength is great because we have to do things one or two or five times, but I've nearly always had people build up endurance so their muscles support them through the day without being excessively fatigued by night time. So we start with light weights, in fact you might start with using bands, but I'm going to show what we progress to, which is free weights. So I'm using just 3 lb. weights which by the way you don't start people that way. It's too heavy for most individuals, but although they will argue with you. Especially a big muscle-bound guy, who will think it's very insulting but they'll learn very quickly. To demonstrate, you bend forward, keep your back flat, knees bent, so that you make it the easiest on your back. By the way, if you're going to bend forward so your back is parallel to the floor you have to suck the stomach up into the rib cage so the back doesn't fatigue and become painful while people are doing this. Okay, so now we're going to change positions and you'll see what we will do. So I'm bent forward, my back parallel to the floor, my knees are bent. My stomach is sucked up into my rib cage so my back doesn't get sore.

The first one we're going to do is a Y. With arms are straight down, you're just going to bring them up, thumbs up. Then lower the arms back down and repeat up to 20 times. When your raise the arms up, you want to come up parallel with your shoulders. That's the Y. The T, again arms are straight down and going to come up to the sides. Now watch, people will often if they're weak, they'll bring the arms back like an arrow and they don't realize it. Have them look to their sides and see that their arms are straight out. So something I should mention the first two of our exercises for trap strength and mainly endurance building, are the Y's and T's as discussed. But what I want to note is the Y, coming up just like a Big Y but bent over, as you know. That is for the upper trap, where the T is specifically to build endurance in the mid trap, right between the shoulder blades. And these are essential to keep the shoulders back and pull the shoulder blades together. Otherwise shoulders are going to roll forward and they'll fatigue very quickly.

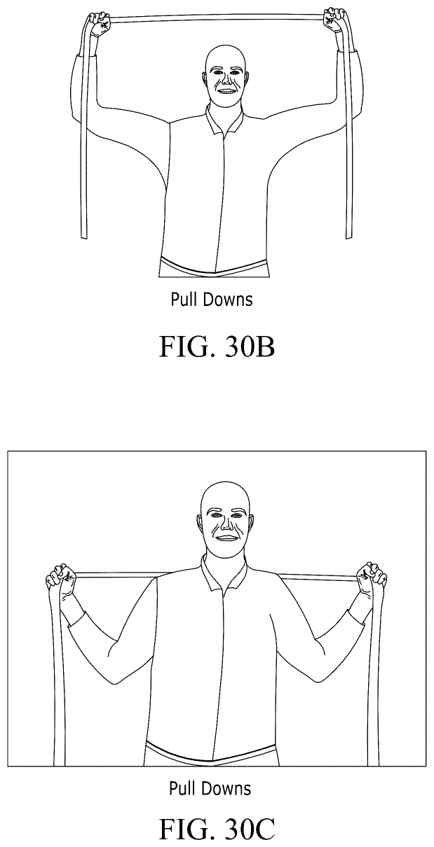

FIGS. 30A-C depict the pull downs stretch, according to various embodiments. This will be started and it's another trap endurance building. Now, I picked the Red Band, you'll probably start with the lighter one, like the yellow or green. Now you want to hold the band about 12-14 inches apart. So, when it's above your head. I guess I'm probably about 14 inches apart. So, as you do it, you're going to bring the elbow straight down to the sides. Don't let them come forward nor back so they're arching their back otherwise people will get a sore back. They're just going to come straight down the side until they hold it down by the side and up. Nice slow, steady movement. So, again, you can see from my arms why it's called a W, that's what it looks like.

The one thing I should mention when doing the W (and this is actually quite important), is when you're overhead, if you only come down to just slightly below the head, it's not enough. This exercise is meant to strengthen and build endurance in the lower traps. If you only come to below the head, you can only feel it just starting to engage. You have to bring the hands down below the tops of the shoulders so your elbows are by your side to feel a full contraction of the lower traps. To get the effectiveness you need, we're going to hold the shoulder blades back in position, which is therefore going to alleviate problems in the traps leading forward. Then you have to make sure the lower traps are in good shape which is extremely important.

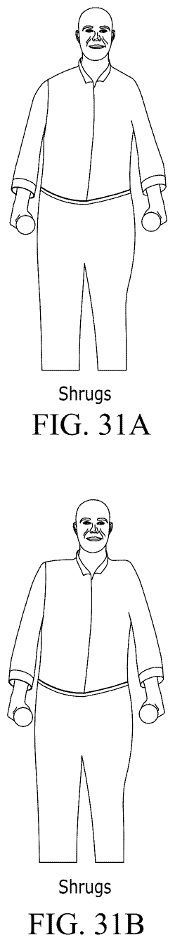

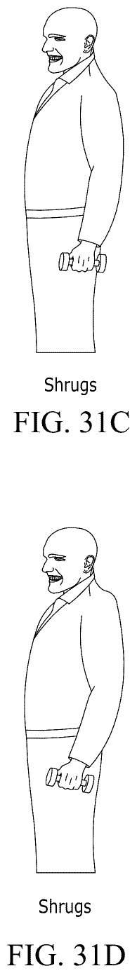

FIGS. 31A-D depict the shrugs stretch, according to various embodiments. Now, we want to build strength in the top of the upper trap. So right across the top of the shoulders basically. Bring the shoulders back. I have 5 pound weights in my hands. Which is low, you're probably going to start maybe 3 or 5 somewhere in that ballpark. Going to keep my shoulders back, chin back, and then raise my shoulders up towards my cars. Hold it for 2 or 3 seconds. Then down but you're keeping your traps and your shoulders back the whole time. Up and down. And people will notice some fatigue along the top throughout their traps. Basically right across the top of the shoulder. So that will help considerably in maintaining shoulder position and also how they hold their shoulders or arms. So now I'm going to do it so you can see my actual shoulder position. You don't want people to have rounded shoulders as they do this because then their shoulders are just going to come up and forward. So you have to exaggerate your chest out, shoulders back and down. Then bring them up towards your cars. Hold, keep shoulders back, and drop them down as low as they can. And let the weights hang as dead weights, you'll feel a good stretch on the way down. Shoulders back, chest out, raise the shoulders up to the cars and let then down. Then exhale, and let the weight pull your shoulders.

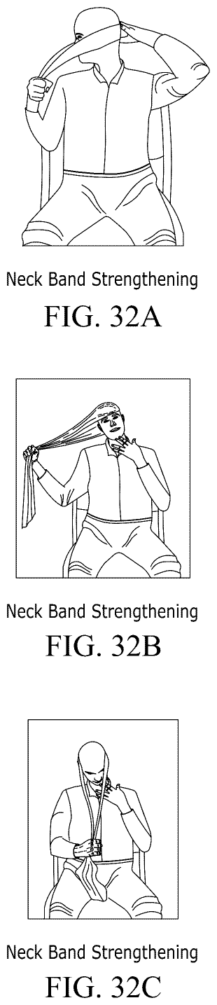

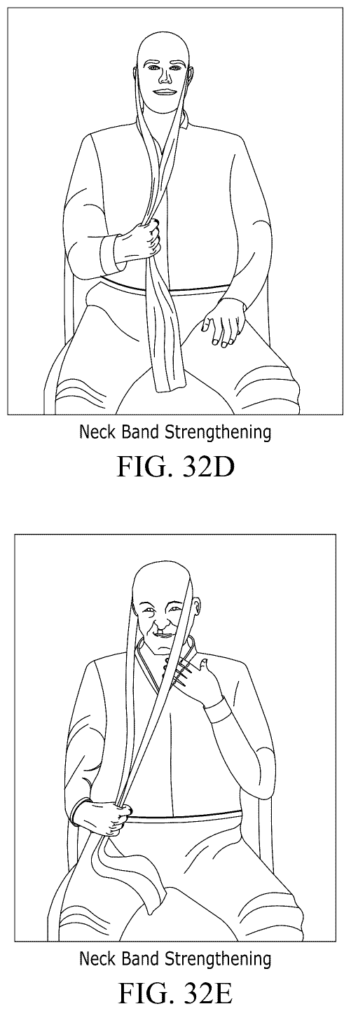

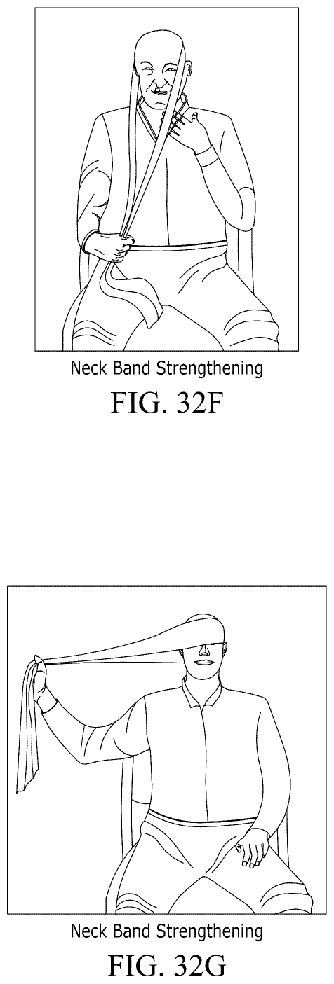

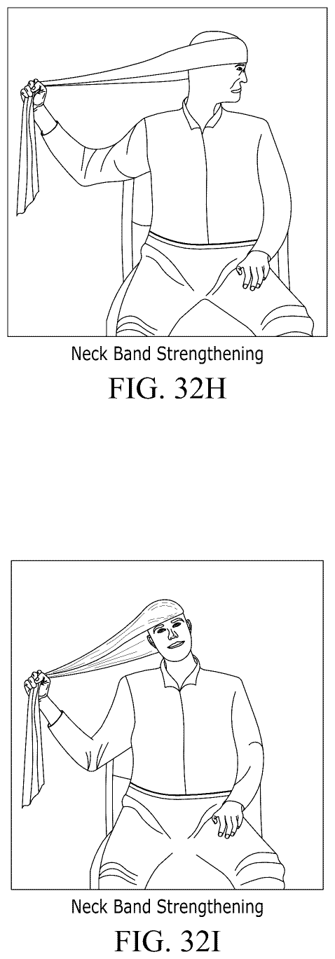

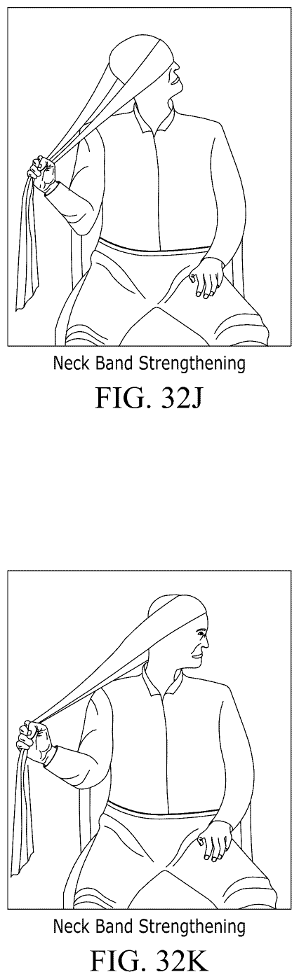

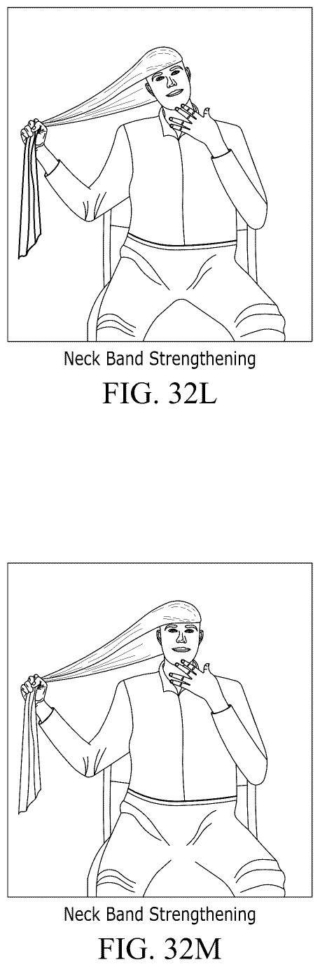

FIGS. 32A-M depict the neck band strengthening exercise, according to various embodiments. It's very important with the stretches to give yourself relief, but you won't get any sustained relief. That's because as you stretch the muscle, the spasm goes, but that's where muscles have been very weak [OR the other muscles will be very weak], so it's going to fatigue and tighten again. Unless you build up the strength and mainly endurance (even more than strength), you seldom progress to getting fully better, it's very limiting. And it's nice to get yourself relief, but the whole point is to get better and to not have to worry about it.

I want to talk about some neck strengthening exercises and the first I'm going to show you is for the back of the neck and up into the sub occipitals. Basically right up by the back of the neck into the base of the skull. But you're going to feel this pull all the way down into the traps, because don't forget the trap also attaches to the base of the skull. So you'll often feel this right down into your shoulder blades, so don't be surprised.

First of all, I'm going to use this red band, now they will usually start with a lower resistance band to start with, but this is something that's comfortable for me. I put the band so it's on the top back of the head. If you have it fully on the back, it's just going to pull down onto your ears and it's not really going to strengthen the neck very much, certainly not the top of the neck. So, you have it on the top back of the head, you retract your chin, and I have tension in the band (moderate tension in the band), holding it with one hand and it's down about almost a 40 degree angle.

I'm going to now let my head come down, but I'm going to maintain resistance. Don't let it suddenly just go slack when you come down. Or, another way you can do it is you start with the head down, but you have to make sure the chin is retracted before you come down. So I can do this, which is probably going to be easier for most of you. I retract my chin, and I tilt my skull down. Then I keep my chin retracted and I bring my head up vertically, it has to be all the way to vertical, then I slowly go back down again. And you can see that where I have this placed, it's not slipping down over my ears, in fact my ears stop it from moving. And my chin is still retracted, it's a lovely picture, you know, double chins. In fact, I actually tease people about it because you want to have double chins if your chin is retracted.

This is the an important [angle], but then you can also do it on either side because you do the stretch with the head rotated to 20 degrees, 40 degrees, and 60 degrees, so you want to strengthen the same. So, again I'm going to retract, keep my chin down, my hand doesn't move. Bring my head up and slowly bring it down. Up to vertical and down. I'm going to do the same thing at 40 degrees, and again at 60. Now, you'll note my hand is staying stable, it's not moving, I'm keeping constant pressure on it and that way you're going to get the same results each time.

A problem some people have is they will try to use the short band or they hold it too close to the head and they have it right up close to their head like this. Well, if you try that, when you come up, you can't get there very well. It's very difficult and it's more likely to slide, so you want to keep it a bit long or you want to use the long band.

The other part is, for those who have lots of hair you want to have the band stay in place, people with longer hair tend to find it slides on their hair. So a lot of people try to use either a toque or a knitted hat or sometimes a baseball cap, it'll work with that on and the band around that and it stays in place easier. With me, I don't have to worry about that, I don't have enough hair to worry about.

Anyways, that's the first one. That's as I said, for the back of the neck and suboccipital and down into the traps. So the next one you want to try, similar idea, again hold it at a distance out from the head. I have my arms locked in place. I have [the band] at the top and side of the head. Start from straight, and I rotate. Slowly, come back. So this gets the sternocleidomastoid or SCM, it's actually one of the easier ones and tends not to slide much. It's not that difficult.

Now, there's also the splenius muscles. That's often forgotten, people remembered to stretch the splenius but you have to strengthen it too or it's going to fatigue and tighten as well. Like the splenius stretch, I'm going to come down to the side, it's actually the same as the stretch, really. You come down to the side, you rotate forward, and I come to about 70 degrees (˜60-70 degrees), and I'll bring (rotate) my ear back over my shoulder. My hand is not moving, it's locked in place. And that's probably one you will want a baseball cap or something like that, because it frequently slides down which is very annoying. You want to keep that chin retracted when you are coming back. People tend to let it out. You want it in place.

We also do the scalene muscle strengthening and it gets RCL (Rectus capitis lateralis) muscle as well. Again, you're going to retract your chin. The band is on the side and top of my head, keep my chin retracted. I start with my head down, my arm locked in place. I come up to vertical and slowly go back down. Up, then down.

None of these are very difficult. They don't take much time. People like the stretches because they feel better and they can't be bothered with the band, but you won't likely get better if you don't strengthen and build endurance in the neck. Now let's face it, the bands aren't that strong, so it's more about endurance than it is about strength.

The phrase “physical disorder” is used throughout this disclosure to explain and discuss the various embodiments. A “physical disorder” is a physical condition that significantly and detrimentally impacts a person's life due to illness, injury or other forms of bodily degradation. The phrase “therapeutic motion” is used throughout this disclosure to explain and discuss the various embodiments. A “therapeutic motion” is a stretching motion (a stretch) or an exercise that targets a particular muscle or muscle group. A “therapeutic motion” is used to treat a physical disorder caused by an impingement condition. The various embodiments disclosed herein entail use of a TM20 biphasic pulse generation device 100. The device is “biphase” inasmuch as each successive pulse it produces in a pulse train is the negative of the previous pulse. For example, the pulse voltage in a pulse train may be: +25V, −25V, +25V, −25V, etc.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises,” “comprising,” “includes,” and/or “including” used in this specification specify the presence of stated features, integers, steps, operations, elements, and/or components, but do not preclude the presence or addition of one or more other features, integers, steps, operations, elements, components, and/or groups thereof. The term “plurality”, as used herein and in the claims, means two or more of a named element. It should not, however, be interpreted to necessarily refer to every instance of the named element in the entire device. Particularly, if there is a reference to “each” element of a “plurality” of elements. There may be additional elements in the entire device that are not be included in the “plurality” and are not, therefore, referred to by “each.”

The description of the various embodiments provided above is illustrative in nature inasmuch as it is not intended to limit the invention, its application, or uses. Thus, variations that do not depart from the intents or purposes of the invention are intended to be encompassed by the various embodiments of the present invention. Such variations are not to be regarded as a departure from the intended scope of the present invention.

What is claimed is:

1. A method of diagnosing and treating a patient with a physical disorder due to an impingement condition, the method comprising:
gather patient medical information including information about illness, injury or other forms of bodily degradation experienced by the patient;
identify a symptom cluster including one or more symptoms of the impingement condition, wherein the symptom cluster includes at least one core symptom;
diagnose a physical disorder based on the patient medical information and the symptom cluster, wherein the physical disorder is caused by the impingement condition;
generate a synthetic stimulatory neural signal sequence to treat the physical disorder;
apply the synthetic stimulatory neural signal sequence to one or more muscles of the patient causing the impingement condition; and
prescribe a least one therapeutic motion to eliminate the impingement condition and eliminate the one or more symptoms of the cluster symptom.

2. The method of claim 1, wherein the impingement condition is a nerve impingement.

3. The method of claim 1, wherein the impingement condition is a vessel impingement.

4. The method of claim 2, wherein the patient medical information includes a patient medical history and a description of the injury related to the impingement condition.

5. The method of claim 2, wherein the synthetic stimulatory neural signal sequence is generated with a biphasic pulse generation device; and
wherein the synthetic stimulatory neural signal sequence is a predefined sequence of timed low current level electrical pulses.

6. The method of claim 5, wherein the synthetic stimulatory neural signal sequence includes a plurality of periods of pulses of low-level current, each of the plurality of periods of pulses having a different frequency.

7. The method of claim 6, wherein the pulses of low-level current are no greater than 25 milliamps; and
wherein the plurality of periods of pulses includes a first period of 80 +/−20 hertz pulses and a second period of 4 +/−2 hertz pulses.

8. The method of claim 7, wherein the at least one therapeutic motion is a stretch.

9. The method of claim 8, wherein the at least one core symptom includes a first core symptom and a second core symptom and the symptom cluster includes at least one condition indicator symptom.

10. The method of claim 9, wherein the physical disorder is post concussion syndrome.

11. The method of claim 9, wherein the physical disorder is glaucoma.

12. The method of claim 9, wherein the physical disorder is Parkinson's disease.

13. An apparatus for treating a patient with a physical disorder due to an impingement condition, the apparatus comprising:
a user interface section configured to receive control inputs from a human user to adjust and operate the apparatus;
a user display section including a plurality of LED indicators;
a controller unit with electronic logic to control functions of the apparatus in response to the control inputs entered by the human user to the user interface section;
one or more signal generators;
a plurality of electrodes electrically connected by one or more stimulation cables to the one or more signal generators; and
an enclosure cabinet;
wherein each of the plurality of electrodes is configured to be temporarily electrically connected to the patient;
wherein the one or more signal generators produces a synthetic stimulatory neural signal sequence to treat the physical disorder, the synthetic stimulatory neural signal sequence including a predefined sequence of timed low current level electrical pulses.

14. The apparatus of claim 13, wherein the synthetic stimulatory neural signal sequence includes a plurality of periods of pulses of low-level current, each of the plurality of periods of pulses having a different frequency.

15. The apparatus of claim 14, wherein the pulses of low-level current are no greater than 25 milliamps; and
wherein the plurality of periods of pulses includes a first period of 80 +/−20 hertz pulses and a second period of 4 +/−2 hertz pulses.

16. The apparatus of claim 15, wherein the physical disorder is post concussion syndrome.

17. The apparatus of claim 15, wherein the physical disorder is glaucoma.

18. The apparatus of claim 15, wherein the physical disorder is Parkinson's disease.
